(19) United States
(12) Patent Application Publication
(10) Pub. No.: US 2019/0133160 A1
(43) Pub. Date: May 9, 2019

(54) CYANOBACTERIOCHROMES AS COLOR-FAST OR COLOR-SWITCHING FOOD ADDITIVES

(71) Applicant: THE REGENTS OF THE UNIVERSITY OF CALIFORNIA, Oakland, CA (US)

(72) Inventors: John Clark Lagarias, Davis, CA (US); Nathan Clarke Rockwell, Davis, CA (US); Shelley S. Martin, Davis, CA (US)

(73) Assignee: THE REGENTS OF THE UNIVERSITY OF CALIFORNIA, Oakland, CA (US)

(21) Appl. No.: 16/191,200

(22) Filed: Nov. 14, 2018

Related U.S. Application Data
(63) Continuation of application No. PCT/US2017/033360, filed on May 18, 2017.
(60) Provisional application No. 62/339,034, filed on May 19, 2016.

(51) Int. Cl. A23L 5/46; C07K 14/195; A23L 2/58; A23G 9/38

(52) U.S. Cl. CPC A23L 5/46; C07K 14/195; A23V 2002/00; A23G 9/38; A23L 2/58

(57) ABSTRACT

Compositions and methods are disclosed for the coloring of foodstuffs with recombinant, purified, and/or isolated cyanobacteriochromes (CBCRs). The CBCRs can affect the apparent color of a food by selective absorbing or fluorescing particular wavelengths of light. The CBCRs can have either colorfast properties, in which the CBCRs consistently appear as having a single color, or color changing properties, in which the CBCRs can switch between two states that each appear as a different color.

### CROSS-REFERENCES TO RELATED APPLICATIONS

The present application is a continuation of International Patent Application No. PCT/US2017/033360, filed May 18, 2017, which claims priority to U.S. Provisional Application No. 62/339,034, filed May 19, 2016, which is incorporated by reference in its entirety herein for all purposes.

### STATEMENT AS TO RIGHTS TO INVENTIONS MADE UNDER FEDERALLY SPONSORED RESEARCH AND DEVELOPMENT

This invention was made with Government support under Grant No. DE-FG02-09ER16117, awarded by the U.S. Department of Energy. The Government has certain rights in this invention.

### REFERENCE TO A SEQUENCE LISTING

The Sequence Listing written in file SequenceListing_070772-223310US-1111911.txt created on Nov. 13, 2018, 188,820 bytes, machine format IBM-PC, MS-Windows operating system, is hereby incorporated by reference in its entirety for all purposes.

### BACKGROUND OF THE INVENTION

Many modern foodstuffs use added color agents. Such agents can be produced via chemical synthesis, via biosynthesis in living cells, or via a combination of these methods. Artificial colors used as food additives include aniline or azo dyes, FD&C dyes approved in the United States, and E dyes approved in the European Union. Natural color agents contain pigments that are synthesized in living cells. For example, the annatto color widely used in cheesemaking derives its coloration from carotenoid pigments. Similarly, the blue-green colored light-harvesting phycobiliprotein phycocyanin contains the linear tetrapyrrole (bilin) chromophore phycocyanobilin (PCB). Phycocyanin is already used as a natural pigment in the food and beverage industries under the trade name LINABLUE® natural blue food coloring, with extensive use in Asia and Indonesia. Other bilins can be found in related light-harvesting phycobiliproteins such as phycoerythrin (pink) and phycourobilin (amber).

Bilins are also used in photoreceptors such as phytochromes and cyanobacteriochromes (CBCRs). Unlike phycocyanin and related light-harvesting biliproteins, bilin-based photoreceptors photoswitch between two states that typically sense different regions of the spectrum (photochromism). For example, CBCR NpR6012g4 senses red light when in the ground state but senses green light after illumination with red light. CBCRs are able to detect a very broad range of colors and hence adopt a broader range of possible hues than phycobiliproteins. CBCRs are present in almost all cyanobacteria, including strains eaten by humans or used for production of food additives such as LINABLUE® or Spirulina extract. For example, Arthrospira platensis (the source of “Spirulina extract” approved by the FDA for coloring candy and chewing gum) has been shown to contain at least one functional CBCR (Rockwell et al. (2015) Photochem. Photobiol. Sci. 14:929-941).

### BRIEF SUMMARY OF THE INVENTION

In general, provided herein are compositions and methods for coloring a food with one or more recombinant and/or isolated (e.g., purified) cyanobacteriochromes (CBCRs).

One provided food composition comprises a food and one or more recombinant CBCRs. Also provided is a food composition comprising a food and one or more isolated or purified CBCRs.

In some embodiments, the one or more phytochromes or CBCRs of a food composition can each independently comprise NpAF142g1, NpAF142g2, NpAF142g3, NpF0020, NpF1000, NpF1183, NpF1883g2, NpF1883g3, NpF1883g4, NpF2164g2, NpF2164g3, NpF2164g4, NpF2164g5, NpF2164g6, NpF2164g7, NpF2854g1, NpF2854g2, NpF2854g3, NpF4973, NpF6001, NpF6362, NpR1060, NpR1597g1, NpR1597g2, NpR1597g4, NpR1759, NpR2903, NpR3691, NpR3784, NpR3797, NpR4776g1, NpR4776g2, NpR4776g3, NpR5113g1, NpR5113g2, NpR5113g3, NpR5313g1, NpR5313g2, NpR6012g2, NpR6012g3, NpR6012g4, Synechocystis Cph1, Synechocystis Cph2, CparGPS1, GwitGPS1, EsPHL1, DtenPHY1, NpyrPHY1, PcolPHY1, TastPHY1, Ava_3771, Anacy_2551g3, Anacy_3174g6, Anacy_4718g3, Apl_4973, WP_016873240, WP_016878855, AFZ15460g3, Cyan7822_4053g2, Cyan8802_2776g1, UYIDRAFT_04680, WP_016871037, M595_0799, Mic7113_2205, Mic7113_2408, LYNGBM3L_56870g6, Nos7524_4790, Fdi_DRAFT46470, Pleur7313DRAFT_05530, WP_033374293, Sta7437_1656, Syn7502_01757, Oscil6304_1286, Oscil6304_4336g2, Oscil6304_4065g2, Oscil6304_4080, Oscil6304_4203, Oscil6304_2705, Oscil6304_3021, Oscil6304_4174, RcaE, or Tlr0924. In some embodiments, the one or more CBCRs of a food composition can each independently comprise NpR6012g4, NpR4776g3, Tlr0924, NpR5113g2, NpF1883g3, NpF2164g3, NpF2164g5, NpF2164g6, Anacy_2551g3 or Anacy_3174g6. In some embodiments, the one or more CBCRs of a food composition can each independently comprise variants of the aforementioned proteins or of related biliprotein photosensors.

In some embodiments, the one or more CBCRs of a food composition comprise one or more truncated CBCR domains. In some embodiments, each of the one or more CBCRs of a food composition independently has a length of less than about 230 amino acids.

In some embodiments, at least one of the one or more CBCRs of a food composition exhibits a color change in response to light comprising a photoconversion wavelength. In some embodiments, at least one of the one or more CBCRs of a food composition exhibits fluorescence in response to light comprising a fluorescence activation wavelength. In some embodiments, at least of the one or more CBCRs of a food composition exhibits a color change in response to a pH having a protochromic pH value.

In some embodiments, a food composition comprises a first CBCR having a light absorption at a first wavelength, and a second CBCR having a light absorption at a second wavelength that is different from the first wavelength. In some embodiments, the food composition also comprises a protein, and the first and second CBCRs are each domains of the protein.

In some embodiments, the food of the food composition is a beverage. In some embodiments, the food of the food composition is a non-beverage. In some embodiments, the food of the food composition can be an ice pop, a frosting, a glaze, a shell, or a coating.

One provided food coloring composition comprises one or more recombinant CBCRs. Also provided is a food coloring composition comprising one or more isolated or purified CBCRs.

In some embodiments, the one or more phytochromes or CBCRs of a food coloring composition can each independently comprise NpAF142g1, NpAF142g2, NpAF142g3, NpF0020, NpF1000, NpF1183, NpF1883g2, NpF1883g3, NpF1883g4, NpF2164g2, NpF2164g3, NpF2164g4, NpF2164g5, NpF2164g6, NpF2164g7, NpF2854g1, NpF2854g2, NpF2854g3, NpF4973, NpF6001, NpF6362, NpR1060, NpR1597g1, NpR1597g2, NpR1597g4, NpR1759, NpR2903, NpR3691, NpR3784, NpR3797, NpR4776g1, NpR4776g2, NpR4776g3, NpR5113g1, NpR5113g2, NpR5113g3, NpR5313g1, NpR5313g2, NpR6012g2, NpR6012g3, NpR6012g4, Synechocystis Cph1, Synechocystis Cph2, CparGPS1, GwitGPS1, EsPHL1, DtenPHY1, NpyrPHY1, PcolPHY1, TastPHY1, Ava_3771, Anacy_2551g3, Anacy_3174g6, Anacy_4718g3, Apl_4973, WP_016873240, WP_016878855, AFZ15460g3, Cyan7822_4053g2, Cyan8802_2776g1, UYIDRAFT_04680, WP_016871037, M595_0799, Mic7113_2205, Mic7113_2408, LYNGBM3L_56870g6, Nos7524_4790, Fdi_DRAFT46470, Pleur7313DRAFT_05530, WP_033374293, Sta7437_1656, Syn7502_01757, Oscil6304_1286, Oscil6304_4336g2, Oscil6304_4065g2, Oscil6304_4080, Oscil6304_4203, Oscil6304_2705, Oscil6304_3021, Oscil6304_4174, RcaE, or Tlr0924. In some embodiments, the one or more CBCRs of a food coloring composition can each independently comprise NpR6012g4, NpR4776g3, Tlr0924, NpR5113g2, NpF1883g3, NpF2164g3, NpF2164g5, NpF2164g6, Anacy_2551g3, or Anacy_3174g6. In some embodiments, the one or more CBCRs of a food composition can each independently comprise variants of the aforementioned proteins or of related biliprotein photosensors.

In some embodiments, the one or more CBCRs of a food coloring composition comprise one or more truncated CBCR domains. In some embodiments, each of the one or more CBCRs of a food coloring composition independently has a length of less than about 230 amino acids.

In some embodiments, at least one of the one or more CBCRs of a food coloring composition exhibits a color change in response to light comprising a photoconversion wavelength. In some embodiments, at least one of the one or more CBCRs of a food coloring composition exhibits fluorescence in response to light comprising a fluorescence activation wavelength. In some embodiments, at least of the one or more CBCRs of a food coloring composition exhibits a color change in response to a pH having a protochromic pH value.

In some embodiments, a food coloring composition comprises a first CBCR having a light absorption at a first wavelength, and a second CBCR having a light absorption at a second wavelength that is different from the first wavelength. In some embodiments, the food coloring composition also comprises a protein, and the first and second CBCRs are each domains of the protein.

Also provided is a method of coloring a food comprising any of the previously described food coloring compositions to a food.

In some embodiments, the food of a food coloring method is a beverage. In some embodiments, the food of a food coloring method is a non-beverage. In some embodiments, the food of a food coloring method is an ice pop, a frosting, a glaze, a shell, or a coating.

In some embodiments, a food coloring method further comprises subsequently illuminating the food with a light comprising a photoconversion wavelength, wherein at least one of the one or more CBCRs exhibits a color change in response to light having the photoconversion wavelength. In some embodiments, a food coloring method further comprises subsequently illuminating the food with a light comprising a photoconversion intensity, wherein at least one of the one or more CBCRs exhibits a color change in response to light having the photoconversion intensity. In some embodiments, a food coloring method further comprises subsequently adjusting the pH of the food to a protochromic pH value, wherein at least one of the one or more CBCRs exhibits a color change in response to a pH having the protochromic pH value.

Other objects, features, and advantages of the present invention will be apparent to one of skill in the art from the following detailed description and figures.

### BRIEF DESCRIPTION OF THE DRAWINGS

FIG. 1 shows the absorption spectrum of the 15Z and 15E states of the NpR6012g4 cyanobacteriochrome (CBCR). In this example of CBCR color switching, NpR6012g4 converts between red- and green-absorbing states. The red-absorbing state is the dark-stable state, and the green-absorbing state is the photoproduct. The photoproduct state of NpR6012g4 is stable for hours to days depending on the temperature.

FIG. 2 shows an example of colorfast CBCRs. CBCRs Tlr0924 (left: yellow, with biliverdin IXα as chromophore) and NpF2164g5 (right: blue, with PCB as chromophore) are shown under white light. Both samples do not change color under white light.

FIG. 3 shows an example of generating additional colors with CBCRs. The samples of FIG. 2 are shown along with a green sample generated by mixing 1 part of each. Mixing and photography were performed under white light without changes in color.

FIG. 4 shows an example of independent CBCR photocycles in a tandem fusion. (top left) Absorption spectra are shown for a 2-domain fragment of the Npun_R5113 protein, comprising NpR5113g2, NpR5113g3, and the naturally occurring linker between the two. Spectra are shown after illumination with 500±20 nm light (solid line), after subsequent illumination with 650±20 nm light (dashed line, filled circles), and after illumination with 650±20 nm light followed by 436±5 nm light (dotted line, empty circles). (top right) Difference spectra are shown for the same construct. Difference spectra were calculated as (before—after illumination) for 650±20 nm (dashed line, filled circles) and 436±5 nm light (dotted line, empty circles). (middle left) The difference spectrum generated with 650±20 nm light (dashed line, filled circles) matches that of isolated NpR5113g2 (solid line). (middle right) The difference spectrum generated with 436±5 nm light (dotted line, empty circles) is similar to that of isolated NpR5113g3 (solid line) but contains additional signals matching the inverted difference spectrum of NpR5113g2. These signals arise because 436±5 nm light has spectral overlap with NpR5113g2, resulting in formation of a photoequilibrium of this domain. (bottom) To confirm that the difference spectrum generated by 436±5 nm light is a simple combination of signals from isolated NpR5113g2 and NpR5113g3, the experimental difference spectrum for the tandem construct (dotted line, empty circles) is compared to a calculated spectrum derived from the difference spectra shown for the isolated domains (solid line, filled squares). The calculated spectrum was calculated as (NpR5113g3−22*NpR5113g2) and gave a mean difference to experiment of 0.0002±0.002 Abs (n=251 points).

FIG. 5 shows photochromism in a model ice pop. (left) NpR6012g4 was incorporated into an ice pop, with green light illumination prior to freezing overnight. Color was retained under these conditions. (right) Photochromism was also retained: a red laser pointer was used to photoswitch a narrow region of the ice pop, resulting in formation of a pink stripe in the ice pop.

FIG. 6 shows preparations of eight different CBCRs in ice pop mix under white light and photographed prior to freezing. Top row: NpR6012g4, NpR4776g3, Tlr0924, NpR5113g2. Bottom row: NpR5113g1, NpF2164g3, NpF1883g3, and NpF2164g6.

FIG. 7 shows the preparations of FIG. 6 after overnight freezing but prior to illumination.

FIG. 8 shows the preparations of FIG. 6 after illumination of NpR4776g3, Tlr0924, NpR5113g2, NpR5113g1, NpF2164g3, NpF1883g3, and NpF2164g6. Pronounced color changes are not seen for several CBCRs. NpR6012g4 was not illuminated. NpF2164g3 yields a change from transparent to blue (left half, not illuminated and clear; right half, brief illumination with violet laser pointer, blue).

FIG. 9 shows additional color changes in model ice pops containing Anacy_2551g3 (top) and Anacy_3174g6 (bottom). Anacy_2551g3 is shown in the dark-stable far-red-absorbing state (appears as pale green, left) and after far-red LEDs (728 nm, Sanyo) were used to generate the orange-absorbing photoproduct state (appears as blue to purple, right). Anacy_3174g6 is shown in the dark-stable green-absorbing state (appears as pink, left) and after a green laser pointer was used to generate the blue-absorbing photoproduct state (appears as yellow, right).

FIG. 10 shows ice pops containing NpF2164g5 (top) and NpR6012g4 (bottom) under white light (left) or ultraviolet transillumination (right). White light produces a mix of the two photostates in NpR6012g4, and ultraviolet light induces only weak red fluorescence. NpF2164g5 instead exhibits intense fluorescence upon transillumination. Fluorescence from NpF2164g5 is also red, but the response of the digital camera was saturated under these conditions.

### DETAILED DESCRIPTION OF THE INVENTION

### I. GENERAL

The inventors have discovered that cyanobacteriochromes (CBCRs) can be used as food coloring agents. Some CBCRs can be used as colorfast pigments that provide a range of colors either singly or in combinations. Such combinations can be generated by mixing isolated pigments or by use of larger protein constructs that contain multiple CBCR domains. Additionally, some CBCRs can be used to make foods or beverages that will change color in response to specific colors or intensities of light, or in response to specific pH changes.

As such, the present invention provides compositions and methods for the coloring of foodstuffs with recombinant, purified, and/or isolated CBCRs. The CBCRs can affect the apparent color of a food by selective absorbing or fluorescing particular wavelengths of light. The CBCRs can have either colorfast properties, in which the CBCRs consistently appear as having a single color, or color changing properties, in which the CBCRs can switch between two states that each appear as different colors. The switching of colors can be caused by illumination of the CBCR with a light having a particular wavelength or intensity, or by altering the pH of the CBCR environment. Different portions, elements, or regions of the food can be colored with CBCRs differently so as to appear as different colors or to have different color switching properties. Multiple CBCRs can be combined to produce different additive colors. The multiple CBCRs can be combined as distinct components of a composition or as distinct domains of a shared larger protein.

### II. DEFINITIONS

As used herein, the terms “cyanobacteriochrome” and “CBCR” refer to sensory photoreceptor proteins that mediate one or more processes including, but not limited to, photochromic responses, protochromic responses, phototactic responses, development, and nitrogen metabolism in cyanobacteria. CBCRs typically include at least one GAF domain and at least one bilin chromophore as described herein. Among other characteristics, CBCRs exhibit a variety of photocycles spanning the entire visible, near-IR, and near-UV spectrum. At least six subfamilies of CBCRs have been identified based on photochemistry and primary structure. Examples of previously known CBCRs include AnPixJg2, TePixJg, CikA, and CcaS.

As used herein, the term “GAF domain” refers to a polypeptide having a characteristic tertiary structure present in a number of cGMP phosphodiesterases, certain adenylate cyclases, and the bacterial transcription factor FhlA as first described by Aravind and Ponting (Aravind & Ponting (1997) Tr. Biochem. Sci. 22(12):458-459). The structure of GAF domains is described, for example, by Hurley et al. (Hurley et al. (2000) EMBO 19:5288-5299) and Narikawa et al. (Narikawa et al. (2013) Proc. Nat. Acad. Sci. USA, 110:918-923). Characteristic features of GAF domains of the invention include a central 5-stranded antiparallel β-sheet, one or more α-helices on the chromophore-binding side of the domain, and two or more α-helices opposite the chromophore-binding side of the domain.

The “bilin” chromophores of the invention are linear oligopyrroles (e.g., di-, tri-, or tetrapyrroles) capable of fluorescing, or photointerconverting between spectrophotometrically distinct forms, when associated with an apoprotein. Typically, the bilin components of the invention are isolated from vascular plants, algae, or cyanobacteria according to standard techniques or are synthesized in the same cell in which a CBCR is expressed. The bilin components can also be synthesized de novo. For a general discussion of bilins useful in the present invention, see, e.g., Falk (1989) Pp. 355-399 In: The Chemistry of Linear Oligopyrroles and Bile Pigments, Springer-Verlag, Vienna. Examples of bilins include, but are not limited to, phycocyanobilin (PCB), phytochromobilin (PΦB), phycoerythrobilin (PEB), and any of the four biliverdin IX (BV) isomers: BV IXα, IXβ, IXγ or IXδ.

Bilins and other tetrapyrrole chromophores can be isolated from natural sources or synthesized according to techniques known in the art. Methods for synthesis of the dimethyl ester of phytochromobilin are described, for example, by Weller et al. (Weller et al. (1980) Chem. Ber. 113:1603-1611). Conversion of the dimethyl ester to the free acid can be accomplished according to known techniques (see, e.g., Greene and Wuts (2007) Protective Groups in Organic Synthesis, 4th Ed., Wiley-Interscience, New York). Bilins including phytochromobilin, phycocyanobilin (PCB), and phycoerythrobilin (PEB) can be isolated from natural sources according to known methods. For instance, crude phycocyanobilin can be prepared from Spirulina platensis as described by Terry et al. (Terry et al. (1993) J. Biol. Chem. 268:26099-26106). Crude phytochromobilin and PEB can be prepared by methanolysis of Porphyridium cruentum cells as described by Cornejo et al. (Cornejo et al. (1992) J. Biol. Chem. 267:14790-14798).

The term “recombinant” when used with reference to a protein refers to a protein prepared via genetic engineering. A recombinant cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. A recombinant nucleic acid is a nucleic acid originally formed in vitro, in general, by the manipulation of nucleic acid, e.g., using polymerases and endonucleases, in a form not normally found in nature. A recombinant protein is made using recombinant techniques such as through the expression of a recombinant nucleic acid or DNA molecule as depicted above.

The terms “isolated” and “purified” refer to material which is substantially or essentially free from components which normally accompany it as found in its native state. Purity and homogeneity are typically determined using analytical chemistry techniques such as polyacrylamide gel electrophoresis or high performance liquid chromatography. A protein or nucleic acid molecule which is the predominant protein or nucleic acid species present in a preparation is substantially purified. Generally, an isolated protein or nucleic acid molecule will comprise more than 80 percent of all macromolecular species present in the preparation. Preferably, the protein is purified to represent greater than 90 percent of all macromolecular species present. More preferably, the protein is purified to greater than 95 percent, and most preferably the protein is purified to essential homogeneity, wherein other macromolecular species are not detected by conventional techniques

As used herein, the term “amino acid” refers to any monomeric unit that can be incorporated into a peptide, polypeptide, or protein. Amino acids include naturally occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally occurring amino acids are those encoded by the genetic code, as well as those amino acids that are later modified, e.g., hydroxyproline, γ-carboxyglutamate and O-phosphoserine. Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), selenocysteine (Sec), pyrrolysine (Pyl), and combinations thereof. Stereoisomers of naturally occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally occurring amino acids. For example, “amino acid analogs” can be unnatural amino acids that have the same basic chemical structure as naturally occurring amino acids (i.e., a carbon that is bonded to a hydrogen, a carboxyl group, an amino group) but have modified side-chain groups or modified peptide backbones, e.g., homoserine, norleucine, methionine sulfoxide, and methionine methyl sulfonium. “Amino acid mimetics” refer to chemical compounds that have a structure that is different from the general chemical structure of an amino acid, but that functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, as described herein, may also be referred to by their commonly accepted single-letter codes.

With respect to amino acid sequences, one of skill in the art will recognize that individual substitutions, additions, or deletions to a peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. The chemically similar amino acid includes, without limitation, a naturally occurring amino acid such as an L-amino acid, a stereoisomer of a naturally occurring amino acid such as a D-amino acid, and an unnatural amino acid such as an amino acid analog, amino acid mimetic, synthetic amino acid, N-substituted glycine, and N-methyl amino acid.

The term “food coloring” as used herein refers to a substance that can be added to foods or foodstuffs to alter the apparent color of at least a portion of the food.

The term “food” as used herein refers to an edible substance to be ingested orally. A food can be, for example, a beverage or a non-beverage. A food can be, for example, a sweet, a confectionary, a nutrient, or a pharmaceutical.

The term “edible” as used herein refers to a substance that can be eaten by animals or humans. The term encompasses substances that are deemed non-toxic and can be orally ingested and tolerated.

The term “color” as used herein refers to a visual characteristic of a material imparted by the ability of the material to absorb, scatter, or emit light at different wavelengths. Properties of color that can be affected by the provided embodiments include hue, chroma, purity, saturation, intensity, vividness, value, lightness, brightness, and darkness.

The term “hue” as used herein refers to the color property defined as the degree to which a color is similar to or different from elements of a color wheel or color spectrum. Pure hues are typically designated as red, orange, yellow, green, blue, and purple or violet.

The terms “chroma” or “saturation” as used herein refers to the color property defined as indicating the purity of a hue related to its dilution by white, gray, or black.

The term “value” as used herein refers to the color property defined as indicating the lightness or darkness of a color.

The term “protochromic pH” as used herein refers to a pH value at which an apparent color change for a color switching CBCR is triggered. The apparent color change can be caused by a change in the protonation state of the color switching CBCR. A protochromic pH value can be within the range from 3.0 to 4.0, from 3.5 to 4.5, from 4.0 to 5.0, from 4.5 to 5.5, from 5.0 to 6.0, from 5.5 to 6.5, from 6.0 to 7.0, from 6.5 to 7.5, from 7.0 to 8.0, from 7.5 to 8.5, from 8.0 to 9.0, from 8.5 to 9.5, from 9.0 to 10.0, from 9.5 to 10.5, from 10.0 to 11.0, from 10.5 to 11.5 or from 11.0 to 12.0. A protochromic pH value can be less than 3.0. The protochromic pH value can be greater than 12.0.

The term “excipient” as used herein refers to any substance, such as a powder or a liquid, that forms a vehicle for an active substance. Excipients can be inert or can modify the properties of the active substance. Excipients can include, for example, diluents, agglutinants, binders, lubricants, carriers, stabilizers, or permeation enhancers. Excipients can affect water activity, viscosity, or other fluid properties of the solution to which they are added. Excipients can reduce or prevent microbial growth or proliferation.

The terms “first” and “second” when used herein with reference to CBCRs, wavelengths, or other elements or properties are simply to more clearly distinguish the two elements or properties and are not intended to indicate order.

The terms “about” and “approximately equal” are used herein to modify a numerical value and indicate a defined range around that value. If “X” is the value, “about X” or “approximately equal to X” generally indicates a value from 0.90X to 1.10X. Any reference to “about X” indicates at least the values X, 0.90X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.10X. Thus, “about X” is intended to disclose, e.g., “0.98X.” When “about” is applied to the beginning of a numerical range, it applies to both ends of the range. Thus, “from about 6 to 8.5” is equivalent to “from about 6 to about 8.5.” When “about” is applied to the first value of a set of values, it applies to all values in that set. Thus, “about 7, 9, or 11%” is equivalent to “about 7%, about 9%, or about 11%.”

### III. DESCRIPTION OF THE EMBODIMENTS

### A. Cyanobacteriochromes

A first embodiment provides a food composition comprising a food and one or more recombinant, purified, and/or isolated cyanobacteriochromes (CBCRs). CBCRs are photosensor proteins that are members of the phytochrome superfamily and are found in cyanobacteria. Like the distantly related phytochromes, CBCRs use 15,16-photoisomerization of bilin chromophores to reversibly photoconvert between two states with distinct spectral and biochemical properties (Rockwell & Lagarias (2010) ChemPhysChem 11:1172-1180; Ikeuchi & Ishizuka (2008) Photochem. Photobiol. Sci. 7:1159-1167). In both CBCRs and phytochromes, the bilin is covalently attached to a conserved cysteine residue via a thioether linkage.

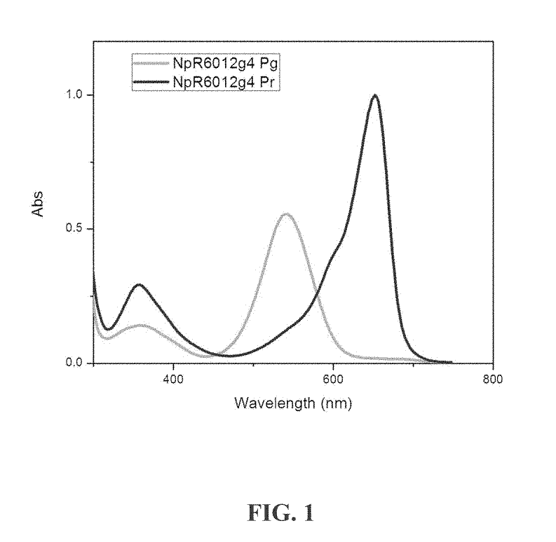

FIG. 1 shows examples of absorption spectra for each of two CBCR states. The example depicted in FIG. 1 is of the 15Z and 15E states of the CBCR NpR6012g4. CBCRs optimally absorb specific wavelengths of light (i.e., different colors) depending on the individual CBCR and on its photostate. Human color vision is also based on differential color absorption by different retinal photoreceptors. As a result, the different light absorbance properties of different CBCRs and different CBCR photostates cause them to appear to adopt different colors to human eyes.

Examples of CBCRs include, but are not limited to, NpAF142g1, NpAF142g2, NpAF142g3, NpF0020, NpF1000, NpF1183, NpF1883g2, NpF1883g3, NpF1883g4, NpF2164g2, NpF2164g3, NpF2164g4, NpF2164g5, NpF2164g6, NpF2164g7, NpF2854g1, NpF2854g2, NpF2854g3, NpF4973, NpF6001, NpF6362, NpR1060, NpR1597g1, NpR1597g2, NpR1597g4, NpR1759, NpR2903, NpR3691, NpR3784, NpR3797, NpR4776g1, NpR4776g2, NpR4776g3, NpR5113g1, NpR5113g2, NpR5113g3, NpR5313g1, NpR5313g2, NpR6012g2, NpR6012g3, NpR6012g4, Synechocystis Cph1, Synechocystis Cph2, CparGPS1, GwitGPS1, EsPHL1, DtenPHY1, NpyrPHY1, PcolPHY1, TastPHY1, Ava_3771, Anacy_2551g3, Anacy_3174g6, Anacy_4718g3, Apl_4973, WP_016873240, WP_016878855, AFZ15460g3, Cyan7822_4053g2, Cyan8802_2776g1, UYIDRAFT_04680, WP_016871037, M595_0799, Mic7113_2205, Mic7113_2408, LYNGBM3L_56870g6, Nos7524_4790, Fdi_DRAFT46470, Pleur7313DRAFT_05530, WP_033374293, Sta7437_1656, Syn7502_01757, Oscil6304_1286, Oscil6304_4336g2, Oscil6304_4065g2, Oscil6304_4080, Oscil6304_4203, Oscil6304_2705, Oscil6304_3021, Oscil6304_4174, RcaE, and Tlr0924.

In particular embodiments, the one or more CBCRs are each independently selected from the group consisting of NpR6012g4, NpR4776g3, Tlr0924, NpR5113g1, NpR5113g2, NpF1883g3, NpF2164g3, NpF2164g5, NpF2164g6, Cyan7822_4053g2, Sta7437_1656, Anacy_2551g3, Anacy_4718g3, Anacy_3174g6, and WP_016871037.

The amino acid sequence of CBCR NpR6012g4 is set forth in SEQ ID NO:1. The amino acid sequence for CBCR NpR4776g3 is set forth in SEQ ID NO:2. The amino acid sequence of CBCR Tlr0924 is set forth in SEQ ID NO:3. The amino acid sequence of CBCR NpR5113g1 is set forth in SEQ ID NO:4. The amino acid sequence of CBCR NpR5113g2 is set forth in SEQ ID NO:5. The amino acid sequence of CBCR NpF1883g3 is set forth in SEQ ID NO:6. The amino acid sequence of CBCR NpF2164g3 is set forth in SEQ ID NO:7. The amino acid sequence of CBCR NpF2164g5 is set forth in SEQ ID NO:8. The amino acid sequence of CBCR NpF2164g6 is set forth in SEQ ID NO:9. The amino acid sequence of CBCR Cyan7822_4053g2 is set forth in SEQ ID NO:10. The amino acid sequence of CBCR Sta7437_1656 is set forth in SEQ ID NO:11. The amino acid sequence of CBCR Anacy_2551g3 is set forth in SEQ ID NO:12. The amino acid sequence of CBCR Anacy_4718g3 is set forth in SEQ ID NO:13. The amino acid sequence of CBCR WP_016871037 is set forth in SEQ ID NO:14. The amino acid sequence of NpR4776g1 is set forth in SEQ ID NO:15. The amino acid sequence of CBCR WP_016878855 is set forth in SEQ ID NO:16. The amino acid sequence of CBCR WP_033374293 is set forth in SEQ ID NO:17. The amino acid sequence of CBCR WP_036801164 is set forth in SEQ ID NO:18. The amino acid sequence of CBCR O6304_4080 is set forth in SEQ ID NO:19. The amino acid sequence of CBCR UYIDRAFT_04680 is set forth in SEQ ID NO:20.

The amino acid sequence of CBCR NpF3797 is set forth in SEQ ID NO:21. The amino acid sequence of CBCR NpF3797 is set forth in SEQ ID NO:22. The amino acid sequence of CBCR WP016873240 is set forth in SEQ ID NO:23. The amino acid sequence of CBCR Nos7524_4790 is set forth in SEQ ID NO:24. The amino acid sequence of CBCR Syn7502_01757 is set forth in SEQ ID NO:25. The amino acid sequence of CBCR NpF1000 is set forth in SEQ ID NO:26. The amino acid sequence of CBCR 06304_4203 is set forth in SEQ ID NO:27. The amino acid sequence of CBCR O6304_4336g2 is set forth in SEQ ID NO:28. The amino acid sequence of CBCR NpR5313g2 is set forth in SEQ ID NO:29. The amino acid sequence of CBCR O6304_3021 is set forth in SEQ ID NO:30. The amino acid sequence of CBCR NpF4973 is set forth in SEQ ID NO:31. The amino acid sequence of CBCR Apl_ORF1 is set forth in SEQ ID NO:32. The amino acid sequence of CBCR O6304_2705 is set forth in SEQ ID NO:33. The amino acid sequence of CBCR NpF1883g2 is set forth in SEQ ID NO:34. The amino acid sequence of CBCR NpF1883g4 is set forth in SEQ ID NO:35. The amino acid sequence of CBCR NpR1597g1B is set forth in SEQ ID NO:36. The amino acid sequence of CBCR NpR5113g3 is set forth in SEQ ID NO:37. The amino acid sequence of CBCR NpAF142g3 is set forth in SEQ ID NO:38. The amino acid sequence of CBCR NpR2903 is set forth in SEQ ID NO:39. The amino acid sequence of CBCR NpF6362 is set forth in SEQ ID NO:40.

The amino acid sequence of CBCR NpR1060 is set forth in SEQ ID NO:41. The amino acid sequence of CBCR NpF6001 is set forth in SEQ ID NO:42. The amino acid sequence of CBCR Mic7113_2205 is set forth in SEQ ID NO:43. The amino acid sequence of CBCR Cyan8802_2776g1 is set forth in SEQ ID NO:44. The amino acid sequence of CBCR NpR3784 is set forth in SEQ ID NO:45. The amino acid sequence of CBCR Ava_3771 is set forth in SEQ ID NO:46. The amino acid sequence of CBCR Mic7113_2408 is set forth in SEQ ID NO:47. The amino acid sequence of CBCR WP_023064634 is set forth in SEQ ID NO:48. The amino acid sequence of CBCR Anacy_3174g6 is set forth in SEQ ID NO:49. The amino acid sequence of CBCR NpF2854g3 is set forth in SEQ ID NO:50. The amino acid sequence of CBCR NpR1597g4 is set forth in SEQ ID NO:51. The amino acid sequence of CBCR NpF2854g1B is set forth in SEQ ID NO:52. The amino acid sequence of CBCR NpF2854g2 is set forth in SEQ ID NO:53. The amino acid sequence of CBCR NpR6012g2 is set forth in SEQ ID NO:54. The amino acid sequence of CBCR NpR6012g3 is set forth in SEQ ID NO:55. The amino acid sequence of CBCR NpAF142g2 is set forth in SEQ ID NO:56. The amino acid sequence of CBCR NpR4776g2 is set forth in SEQ ID NO:57. The amino acid sequence of CBCR NpR4776g2 is set forth in SEQ ID NO:58. The amino acid sequence of CBCR NpF2164g7B is set forth in SEQ ID NO:59. The amino acid sequence of CBCR Moorea_g6 is set forth in SEQ ID NO:60.

The amino acid sequence of CBCR NpR3691 is set forth in SEQ ID NO:61. The amino acid sequence of CBCR fdiDRAFT46470 is set forth in SEQ ID NO:62. The amino acid sequence of CBCR NpR1597g2 is set forth in SEQ ID NO:63. The amino acid sequence of CBCR NpF2164g2 is set forth in SEQ ID NO:64. The amino acid sequence of CBCR 06304_4174 is set forth in SEQ ID NO:65. The amino acid sequence of CBCR 06304_4065g2 is set forth in SEQ ID NO:66. The amino acid sequence of CBCR 06304_1286 is set forth in SEQ ID NO:67. The amino acid sequence of CBCR AFZ15460g3 is set forth in SEQ ID NO:68. The amino acid sequence of CBCR NpF0020 is set forth in SEQ ID NO:69. The amino acid sequence of CBCR NpF1183 is set forth in SEQ ID NO:70. The amino acid sequence of CBCR NpR1759 is set forth in SEQ ID NO:71. The amino acid sequence of CBCR NpR5313g1 is set forth in SEQ ID NO:72. The amino acid sequence of CBCR NpAF142g1 is set forth in SEQ ID NO:73. The amino acid sequence of CBCR Cph1 is set forth in SEQ ID NO:74. The amino acid sequence of CBCR Cph2 is set forth in SEQ ID NO:75. The amino acid sequence of CBCR CparGPS1 is set forth in SEQ ID NO:76. The amino acid sequence of CBCR GwitGPS1 is set forth in SEQ ID NO:77. The amino acid sequence of CBCR EsPHL1 is set forth in SEQ ID NO:78. The amino acid sequence of CBCR DtenPHY1 is set forth in SEQ ID NO:79. The amino acid sequence of CBCR NpyrPHY1 is set forth in SEQ ID NO:80. The amino acid sequence of CBCR PcolPHY1 is set forth in SEQ ID NO:81. The amino acid sequence of CBCR TastPHY1 is set forth in SEQ ID NO:82.

The CBCRs described herein can be obtained after recombinant expression in a host organism. A suitable host organism can be a cyanobacteria such as Arthrospira platensis, an engineered recombinant bacteria such as Escherichia coli, or other organism. E. coli is a well-established source for recombinant proteins that are generally regarded as safe for human use, such as human insulin (FDA approval in 1982). Additionally, the biosynthesis of various bilin chromophores has been demonstrated through engineering of this organism (Gambetta & Lagarias (2001) Proc. Nat. Acad. Sci. USA 98:10566-10571).

In some embodiments, the recombinant host cells are eukaryotic cells. In some embodiments, the recombinant host cells are fungal cells. In some embodiments, the recombinant host cells are prokaryotic cells. In some embodiments, the recombinant host cells are E. coli cells. Suitable prokaryotic host cells include E. coli K12 strain 94 (ATCC No. 31,446), E. coli strain W3110 (ATCC No. 27,325), E. coli K12 strain DG116 (ATCC No. 53,606), E. coli X1776 (ATCC No. 31,537), and E. coli B; and other strains of E. coli, such as LMG194, HB101, JM101, NM522, NM538, and NM539 Plasmids typically used for transformation of E. coli include pBR322, pUCI8, pUCI9, pUCIl8, pUC119, and Bluescript M13, all of which are described in sections 1.12-1.20 of Sambrook et al., Molecular Cloning: A Laboratory Manual (Cold Spring Harbor Laboratory Press, New York, N.Y., 2nd ed. 1989). However, many newer expression vectors are available as well.

CBCRs can be expressed and purified either in the form of a signaling protein that one or more CBCRs is a component of, or in the form of CBCR domains that are isolated from the larger protein. In some embodiments, expression of CBCR domains involves amplifying regions of interest from genomic DNA, cloning the amplification products into an expression vector, and transforming a recombinant host cell with the expression vector. In some embodiments, purification of CBCR domains involves one or more steps of extraction, precipitation, centrifugation, chromatography, lyophilization, or filtration. Exemplary protocols for expression and purification of CBCRs NpR6012g4, NpR4776g3, Tlr0924, NpR5113g1, NpR5113g2, NpF1883g3, NpF2164g3, NpF2164g5 and NpF2164g6 as truncated CBCR domains (under 230 amino acids) have been described (see, e.g., Rockwell et al. (2011) Proc. Nat. Acad. Sci. USA 108:11854-11859; Rockwell et al. (2012) Biochemistry 51:1449-1463; Rockwell et al. (2012) Biochemistry 51:9667-9677).

In some embodiments, the CBCRs used in the provided compositions and methods are recombinant. In some embodiments, the CBCRs used in the provided compositions and methods are purified. In some embodiments, the CBCRs used in the provided compositions and methods are isolated. In some embodiments, the CBCRs used in the provided compositions and methods are recombinant and purified. In some embodiments, the CBCRs used in the provided compositions and methods are recombinant and isolated. In some embodiments, the CBCRs used in the provided compositions and methods are isolated and purified. In some embodiments, the CBCRs used in the provided compositions and methods are recombinant, purified, and isolated.

Most types of phytochromes contain a GAF domain (cGMP-specific phosphodiesterases, adenylate cyclases, and formate hydrogen lyase transcription activator FhlA) that is part of a knotted tridomain PAS-GAF-PHY photosensory core module (Wagner et al. (2005) Nature 438:325-331; Essen et al. (2008) Proc. Nat. Acad. Sci. USA 105:14709-14714; Yang et al. (2008) Proc. Nat. Acad. Sci. USA 105:14715-14720). GAF domains that are isolated from the adjacent domains of these phytochromes are typically insufficient for stable and reversible photoconversion. In contrast, the GAF domains found in CBCRs are typically fully competent by themselves. Several examples have shown that chromophore assembly, peak absorption, and photoconversion are properties of isolated CBCR domains rather than of the diverse cyanobacterial signaling proteins in which such domains are found (Yoshihara et al. (2004) Plant Cell Physiol. 45:1729-1737; Yoshihara et al. (2006) Biochemistry 45:3775-3784; Hirose et al. (2008) Proc. Nat. Acad. Sci. USA 105:9528-9533; Rockwell, et al. (2008) Biochemistry 47:7304-7316; Rockwell et al. (2012) Biochemistry 51:1449-1463; Hirose et al. (2013) Proc. Nat. Acad. Sci. USA 110:4974-4979; Rockwell et al. (2015) Photochem. Photobiol. Sci. 14:258-269). As a result, some embodiments use one or more CBCR domains that are each only a single bilin-binding GAF domain. These CBCR domains can each independently have a length of less than 350 amino acids, less than 320 amino acids, less than 290 amino acids, less than 260 amino acids, less than 230 amino acids, less than 200 amino acids, less than 170 amino acids, or less than 140 amino acids. In some embodiments, the CBCR domains each independently have lengths of less than 230 amino acids.

Some CBCRs do not exhibit color changes upon illumination and hence can be referred to as colorfast agents. For example, CBCR NpF2164g5 does not photoconvert (Rockwell et al. (2012) Biochemistry 51:9667-9677), allowing its use as a colorfast agent. Some CBCRs do exhibit color changes upon illumination and can be referred to as color switching agents. As non-limiting examples, CBCR NpR6012g4 reversibly converts between blue-green and pink colors, and CBCR NpF2164g3 reversibly converts between transparent and blue colors. Anacy_3174g6 reversibly converts between green-absorbing and blue-absorbing photostates that appear pink and yellow, respectively. Anacy_2551g3 reversibly converts between far-red-absorbing and orange-absorbing photostates that appear pale green and blue, respectively. Both proteins have been successfully used in model ice pops. CBCRs not explicitly disclosed herein can also be used in the provided compositions and methods by themselves or in combination with other CBCRs or related photoproteins to generate color changes.

Color switching CBCRs exhibit a color change that can be caused by an isomerization of a thioether-linked bilin chromophore within the GAF domain. The isomerization for a particular color switching CBCR can be triggered by absorption of light having a particular wavelength, referred to herein as a photoconversion wavelength. The spectral diversity of CBCRs and phytochromes is such that the full spectrum of light from near infrared to near ultraviolet can be sensed by some member of the family. The photoconversion wavelength for a CBCR used with the provided compositions and methods can be within the range from 325 nm to 390 nm, from 350 nm to 400 nm, from 375 nm to 425 nm, 400 nm to 450 nm, from 425 nm to 475 nm, from 450 nm to 500 nm, from 475 nm to 525 nm, from 500 nm to 550 nm, from 525 nm to 575 nm, from 550 nm to 600 nm, from 575 nm to 625 nm, from 600 nm to 650 nm, from 625 nm to 675 nm, from 650 nm to 700 nm, from 675 nm to 725 nm, from 700 nm to 750 nm, from 725 to 775 nm, or from 750 nm to 800 nm. The photoconversion wavelength can be greater than 800 nm.

Some CBCRs exhibit fluorescence in one or more of a variety of hues (Rockwell et al. (2012) Biochemistry 51:9667-9677; Rockwell et al. (2008) Biochemistry 47:7304-7316). For example, CBCR NpF2164g5 exhibits intense red fluorescence upon excitation with ultraviolet transillumination. The fluorescent excitation of a particular fluorescent CBCR is triggered by absorption of light having a particular wavelength, referred to herein as a fluorescence excitation wavelength. The fluorescence excitation wavelength for a CBCR used with the provided compositions and methods is typically but not always that of violet or ultraviolet light. The fluorescence excitation wavelength can be within the range from 250 nm to 300 nm, from 275 nm to 325 nm, from 300 nm to 350 nm, from 325 nm to 375 nm, or from 350 nm to 400 nm. The fluorescence excitation wavelength can be greater than 400 nm.

Some CBCRs and phytochromes have a dark-stable state and a photoproduct state that will relax to the dark-stable state in the absence of light. This dark reversion can happen in seconds or over days or weeks. In CBCRs exhibiting dark reversion in seconds, the photoproduct state decays too rapidly to undergo photoconversion. In such cases, photoconversion of the dark state can be driven by much broader wavelength ranges and even by white light, with the amount of color change determined by light intensity rather than by light color (Rockwell et al. (2012) Biochemistry 51:9667-9677. Such CBCRs therefore can be used as coloring agents that will change color in response to light intensity. As a non-limiting example, the orange-absorbing CBCR NpF2164g7 appears blue to human vision and forms an unstable photoproduct that decays in seconds. Photoconversion with white light, orange light, or a range of other colors results in formation of a green-absorbing photoproduct that appears pink to human vision, and the observed amount of photoconversion is linear with applied light intensity. Use of NpF2164g7 in the provided compositions and methods can thus exhibit a blue color in darkness that would shift to purple or pink in the light, with the photoequilibrium and apparent hue determined by light intensity such that brighter light will produce more of the pink photostate.

Some CBCRs respond to both light and pH (Hirose et al. (2013) Proc. Nat. Acad. Sci. USA 110:4974-4979). In these cases, photoconversion can trigger a change in chemical configuration of the chromophore followed by a change in its protonation state, with the latter triggering a substantial color change. Such proteins could therefore be used as coloring agents that would respond to both light and pH. As a non-limiting example, such a CBCR could be combined with a solution of a different pH to trigger one color change, and then light could be used to generate a second color change. The apparent color change for a particular color switching CBCR can be triggered by an adjustment of pH to a particular pH value, such pH value is referred to herein as a “protochromic pH” value. The protochromic pH value for a CBCR used with the provided compositions and methods can be within the range from 3.0 to 4.0, from 3.5 to 4.5, from 4.0 to 5.0, from 4.5 to 5.5, from 5.0 to 6.0, from 5.5 to 6.5, from 6.0 to 7.0, from 6.5 to 7.5, from 7.0 to 8.0, from 7.5 to 8.5, from 8.0 to 9.0, from 8.5 to 9.5, from 9.0 to 10.0, from 9.5 to 10.5, from 10.0 to 11.0, from 10.5 to 11.5 or from 11.0 to 12.0. The protochromic pH value can be less than 3.0. The protochromic pH value can be greater than 12.0.

The CBCRs described herein can be used either individually or in combination with one another. Combined use of two or more CBCRs can be used to produce a color or hue not represented by any of the individual CBCRs in isolation. As non-limiting examples, a red CBCR and a blue CBCR can be used in combination to produce a purple color, a red CBCR and a green CBCR can be used in combination to produce a brown color, a yellow CBCR and a red CBCR can be used in combination to produce an orange color, a yellow CBCR and a blue CBCR can be used in combination to produce a green color, and a yellow CBCR and a red CBCR can be used in combination to produce an orange color. The number of different CBCRs that can be combined can be two or more, three or more, four or more, five or more, six or more, seven or more, eight or more, nine or more, ten or more, eleven or more, twelve or more, or more than twelve. In some embodiments, each of the different combined CBCRs has a different absorbance wavelength. In some embodiments, two or more of the different combined CBCRs have an identical or similar absorbance wavelength in one or both photostates.

Combined use of two of more CBCRs in the provided compositions and methods can involve colorfast CBCRs or color changing CBCRs. As a non-limiting example, a first CBCR converting between green-absorbing and blue-absorbing photostates would appear pink and yellow, respectively, in the two photostates. Use of such a protein by itself would result in conversion between yellow and pink colors. Alternatively, such a protein could be combined with NpF2164g3, which has violet-absorbing and orange-absorbing states that appear transparent and blue, respectively. The violet absorption band of the first CBCR and the blue absorption band of NpF2164g3 overlap, allowing conversion of both states with violet light. The result would then be a switching between yellow (by combining the yellow color of the first CBCR and the transparent color of NpF2164g3) and purple (by combining the pink color of the first CBCR and the blue color of NpF2164g3) states.

Many CBCRs naturally occur as part of larger proteins having multiple CBCRs arranged in tandem (Rockwell et al. (2011) Proc. Nat. Acad. Sci. USA 108:11854-11859). These individual CBCR domains can be isolated from the larger proteins while retaining their functional abilities. Alternatively, one or more recombinant, isolated, and/or purified CBCRs can be combined within a larger recombinant protein such that each of the one or more CBCRs is a domain of this larger protein. In these embodiments, this larger protein fusion construct is then an element of the provided compositions or methods. The number of different CBCRs that can be combined within a larger protein can be two or more, three or more, four or more, five or more, six or more, seven or more, eight or more, nine or more, ten or more, eleven or more, twelve or more, or more than twelve. In some embodiments, each of the different CBCRs combined within the protein has a different absorbance wavelength. In some embodiments, two or more of the different CBCRs combined within the protein have an identical or similar absorbance wavelength.

In some embodiments, the CBCR protein fusion constructs of the present invention are produced by culturing a host cell transformed with an expression vector containing a nucleic acid encoding the two or more CBCRs, under the appropriate conditions to induce or cause expression of the protein fusion containing the CBCRs. Methods of culturing transformed host cells under conditions suitable for protein expression are well known in the art (see, e.g., Sambrook et al., supra). Suitable host cells for production of the CBCR protein fusion constructs from T7 promoter-containing plasmid vectors include E. coli strain BL21 (DE3) and related lysogens (see, e.g., U.S. Pat. No. 5,693,489). Following expression, a CBCR protein fusion construct can be harvested and isolated.

Ongoing surveys of CBCR and phytochrome diversity may isolate colors in addition to those described here. CBCRs not explicitly presented herein can nevertheless be used as coloring agents, as can related photosensors such as phytochromes. As a non-limiting example, orange-absorbing CBCRs such as NpF2164g7 (Rockwell et al. (2012) Biochemistry 51:9667-9677) or orange-absorbing phytochromes from prasinophyte algae (Rockwell et al. (2014) Proc. Nat. Acad. Sci. USA 111:3871-3876) can be used to generate blue colors similar to those seen in NpF2164g3 upon violet illumination. CBCRs and phytochromes not explicitly shown herein can also be used in combination with each other or with CBCRs shown here to generate different colors. As a non-limiting example, combination of colorfast pink and blue CBCRs would generate a colorfast purple in the same way that combination of colorfast blue and yellow CBCRs generates a colorfast green.

### B. Food Compositions

The food that is colored can be a beverage or a non-beverage. In some embodiments, the food is an ice pop, such as a POPSICLE®. In some embodiments, the food is an ice cream. In some embodiments, the food is a frosting, a glaze, a shell, or a coating. The coating can be in the form of one or more coating layers. In some embodiments, the coating further comprises one or more sugars or sugar syrups. The food that is colored can be colored substantially uniformly, or a portion of the food can be colored. In some embodiments, different portions, elements, or regions of the food are colored differently. In some embodiments, one or more portions or elements of the food are colored, and one or more other portions or elements of the food are not colored. Different portions or elements of the food can be colored identically, similarly, or differently. The coloring of the entire food or a portion or element of the food can be with one or more colorfast CBCRs, one or more color changing CBCRs, or both.

### C. Food Coloring Compositions

Also provided is a food coloring composition comprising one or more CBCRs that are recombinant, purified, and/or isolated. The CBCRs can be any of those described above. The food coloring composition can be in the form of a solid, a liquid, a solution, a dispersion, or a mixture of one or more of these. The food coloring composition can further comprise an excipient. The excipient can be selected from known food-grade and/or pharmaceutically acceptable substances that are known to not be toxic to the animal or human ingesting the substance at the dosages and concentrations used. In some embodiments, the excipient is propylene glycol. The excipient can be present in the composition in an amount from 1 wt % to 99 wt %, from 1 wt % to 50 wt %, from 50 wt % to 99 wt %, from 1 wt % to 25 wt % from 25 wt % to 50 wt %, from 50 wt % to 75 wt % from 75 wt % to 99 wt %, from 1 wt % to 10 wt %, from 10 wt % to 20 wt %, from 20 wt % to 30 wt %, from 30 wt % to 40 wt %, from 40 wt % to 50 wt %, from 50 wt % to 60 wt %, from 60 wt % to 70 wt %, from 70 wt % to 80 wt %, from 80 wt % to 90 wt %, or from 90 wt % to 99 wt %. The one or more CBCRs can be present in the composition from 1 wt % to 99 wt %, from 1 wt % to 50 wt %, from 50 wt % to 99 wt %, from 1 wt % to 25 wt % from 25 wt % to 50 wt %, from 50 wt % to 75 wt % from 75 wt % to 99 wt %, from 1 wt % to 10 wt %, from 10 wt % to 20 wt %, from 20 wt % to 30 wt %, from 30 wt % to 40 wt %, from 40 wt % to 50 wt %, from 50 wt % to 60 wt %, from 60 wt % to 70 wt %, from 70 wt % to 80 wt %, from 80 wt % to 90 wt %, or from 90 wt % to 99 wt %.

### D. Methods

Also provided is a method for coloring a food, wherein the method comprises adding any of the food coloring compositions described above to a food. The method can be used to color the entirety of a food, substantially all of a food, or one or more portions of the food. The method can be used to color all or at least a portion of one or more elements of a food. For example, one or more of a coating, a shell, or an edible insert of the food can be colored. In some embodiments, the method alters the hue of a color at least a portion of the food. In some embodiments, the method alters the saturation or chroma of a color of at least a portion of the food. In some embodiments, the method alters the lightness or value of a color of at least a portion of the food. In some embodiments, the change in color is the result of changes in selective color absorption. In some embodiments, the change in color is the result of changes in selective color fluorescence.

The method of coloring a food can be with the use of one or more colorfast CBCRs, one or more color changing CBCRs, or both. In some embodiments, the method comprises adding one or more color changing CBCRs to a food and subsequently illuminating the food with one or more lights having one or more photoconversion wavelengths specific to the one or more color changing CBCRs. In some embodiments, the method comprises adding one or more color changing CBCRs to a food and subsequently illuminating the food with a light having a photoconversion intensity specific to the one or more color changing CBCRs. In some embodiments, the method comprises adding one or more color changing CBCRS to a food and subsequently altering the pH of the food to a protochromic pH value specific to the one or more color changing CBCRs.

The color changing CBCRs can be in one or more portion, region, or element of the food. In some embodiments, different regions of the food are arranged according to a pattern. In some embodiments, the regions form stripes, blocks, circles, or other shapes within the food or on one or more surfaces of the food. Different regions of the food can be colored such that at least one region is a different color that another region. Different regions of the food can be colored such that at least one region is colored with a color changing CBCR and at least one other region is colored with a colorfast CBCR. In some embodiments, different regions of the food are colored with CBCRs such that in a first state two or more regions appear as having the same color, while in a second state the two or more regions appear as having a different color. The color change between the two states can be the result of a change in illumination of the food or a change in the pH of the food.

In certain instances, one or more CBCRs can be activated, enhanced or photoconverted by the application of light of an activating wavelength. They can be inactivated, or their activity can be reduced by the absence of light or by the application of light of an inactivating wavelength. Some CBCRs can be active or show enhanced activity in the dark or reduced light, and can be inactivated or show reduced activity when light of an inactivating wavelength is applied. The “absence of light” can mean the absence of all light (i.e., darkness), or can mean the absence of light in a selected wavelength range that causes a change in the conformation of the CBCR protein module.

It is to be understood that the terms “active” and “inactive” in the foregoing explanation are relative and include complete activity of the protein to complete inactivity of the protein (complete “on/off” modes) as well as relative activity or inactivity of the proteins, i.e., the fusion protein constructs can have high activation ratios, low activation ratios, or activation ratios between high and low. In some embodiments, the fusion protein constructs can be controlled by light so as to have relatively high ratios (e.g., about 2:1 or greater, about 5:1, or about 10:1 or greater) of activity to inactivity or of inactivity to activity under the control of light of appropriate wavelengths.

In addition to applications in foodstuffs, CBCRs provide a palette of repeatedly reversible, color-changing pigments that could be applied to other consumer goods. As non-limiting examples, CBCRs could be used in inks, paints, dyes, or cosmetics such as color-changing eyeliners or nail polishes.

### IV. EXAMPLES

The following examples are offered to illustrate, but not to limit, the claimed invention.

### Example 1

### Different Hues Produced by CBCRs Alone and in Combination

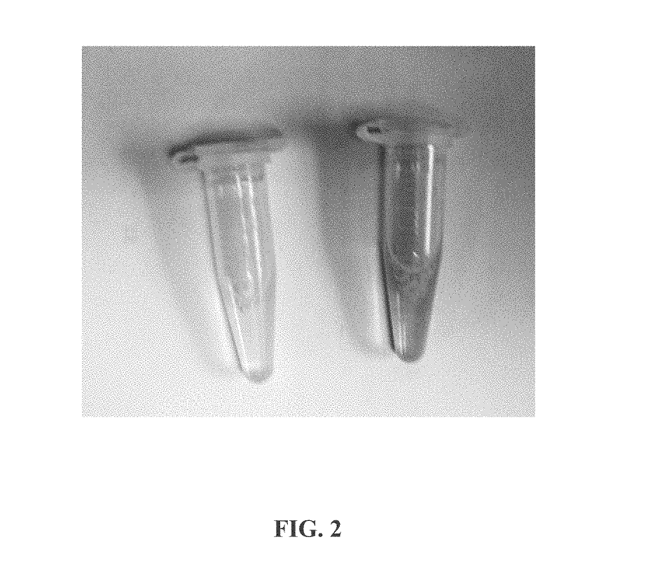

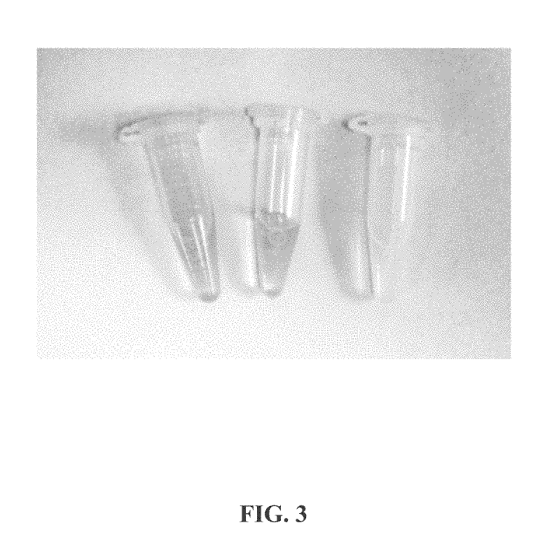

FIG. 2 shows two CBCRs of different colors (NpF2164g5 incorporating PCB chromophore and Tlr0924 incorporating the non-physiological bilin chromophore biliverdin IXα). FIG. 3 shows the same two samples along with a third in which the two were mixed to generate a third color. All sample handling was performed under normal fluorescent lighting, demonstrating that these particular examples do not change color upon illumination. These data demonstrate that CBCRs can provide a range of hues that are stable under normal lighting and that, by mixing multiple CBCRs together, additional colors can be generated.

### Example 2

### Combination of CBCRs in a Recombinant Fusion Protein

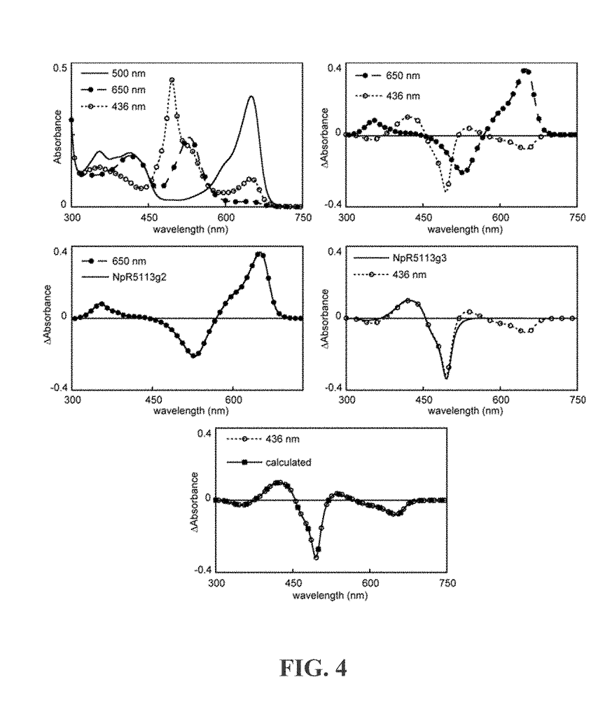

Many CBCRs naturally occur as part of larger proteins having multiple CBCRs arranged in tandem. FIG. 4 shows that the photochemical difference spectra of such tandem arrays are equivalent to those of the isolated domains, indicating that combining CBCRs into a single fusion protein of multiple hues will not cause changes in the observed colors relative to the individual isolated domains.

### Example 3

### Ice Pop Preparation with Colorfast and Color Changing CBCRs

Small ice pops are prepared by mixing 2 ml CBCR solution in various dilute buffers with 2 ml of a premix solution in a small ice cube tray. Premix solution is prepared with 1 part 0.1 M sodium citrate/citric acid, pH 4.9; 1 part 50% glycerol (v/v); 3 parts 50% sucrose (w/v). CBCRs can be stored at −80° C. for months prior to use, and different storage buffers have no effect on the behavior of NpR6012g4 in ice pop mix. After photography, the tray is wrapped in aluminum foil and placed in a −20° C. lab freezer overnight. Samples are then removed and unwrapped. Pictures are taken before and after light treatment. Light treatments use red, green, and/or violet laser pointers. Photographs are taken on standard digital cameras.

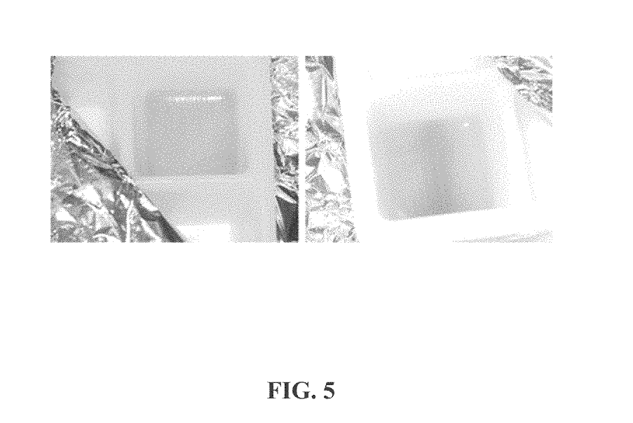

FIG. 5 shows ice pops prepared with NpR6012g4 and illuminated with green light prior to freezing. The left panel shows the dark-stable red-absorbing state of the protein, which can also be regenerated with green light and which appears blue to blue-green to human vision. The right panel shows a pink stripe drawn on the same popsicle by brief localized illumination with a red laser pointer. These data show that CBCRs can be used as color-changing food additives.

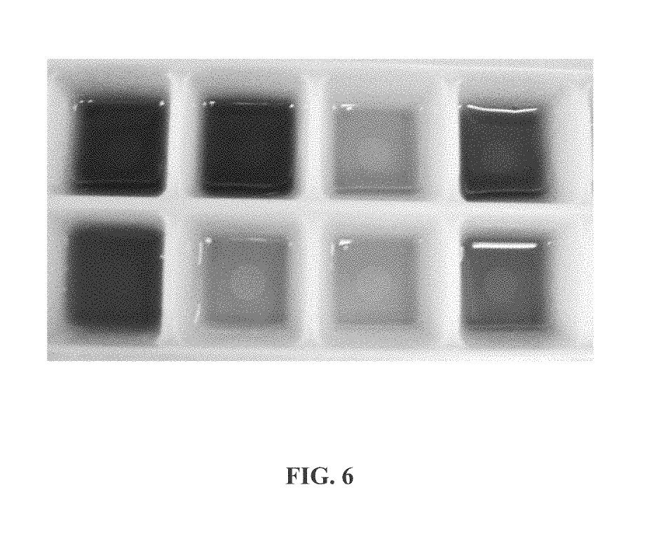

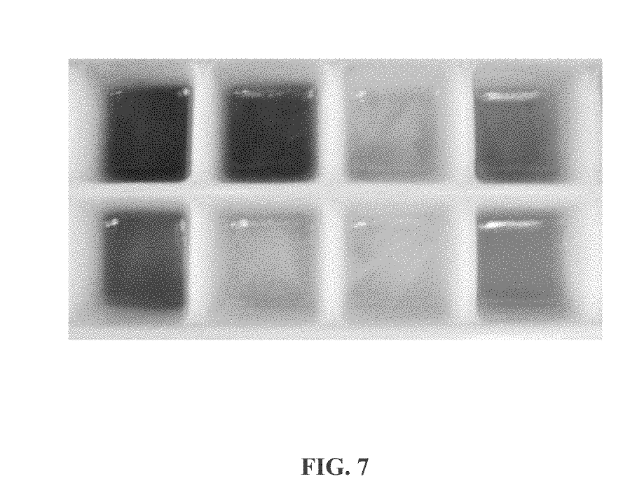

FIG. 6 shows eight CBCRs prepared as ice pops prior to freezing. Top row, left to right: NpR6012g4, NpR4776g3, Tlr0924, and NpR5113g2. Bottom row: NpR5113g1, NpF2164g3, NpF1883g3, and NpF2164g6. FIG. 7 shows the same tray after freezing. In this case, CBCRs were frozen without illumination (at photoequilibrium). NpR6012g4 and NpR4776g3 contain large amounts of both red- and green-absorbing states (cyan and pink, respectively), resulting in an apparent purple color. These data show that a range of CBCRs can be used as coloring agents, giving different hues.

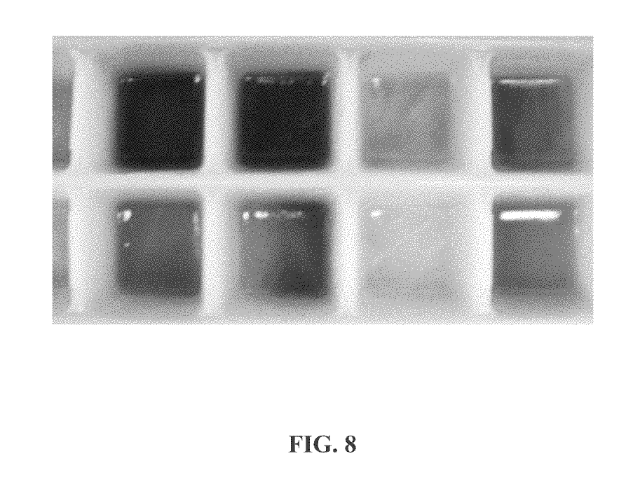

FIG. 8 shows the same tray after illumination of all samples except NpR6012g4. NpR4776g3, NpR5113g2, and NpF2164g6 all show reduced color change compared to NpR6012g4 (red laser pointer). NpR5113g1, NpF1883g3, and Tlr0924 show only minor color change at this temperature and pH (green, violet, and violet laser pointer, respectively). These proteins thus can be used as colorfast agents under these conditions. NpF2164g3 demonstrates a remarkable color change (from transparent to deep blue) upon illumination with the violet laser pointer. These data demonstrate that some but not all CBCRs are color-changing food additives that can generate different colors upon illumination.

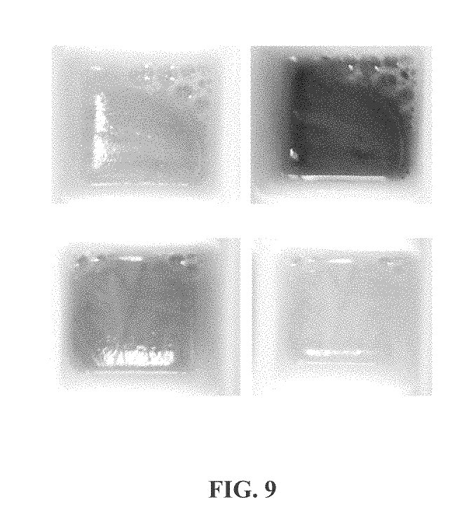

FIG. 9 shows model ice pops containing Anacy_2551g3 (top) or Anacy_3174g6 (bottom). Both proteins are shown before (left) and after (right) illumination with appropriate wavelengths of light to trigger color changes: Anacy_2551g3, far-red light; Anacy_3174g6, green light. Both CBCRs are color changing, providing further examples of the different color changes that are possible with different CBCRs.

### Example 4

### CBCRs as Fluorescent Food Additives

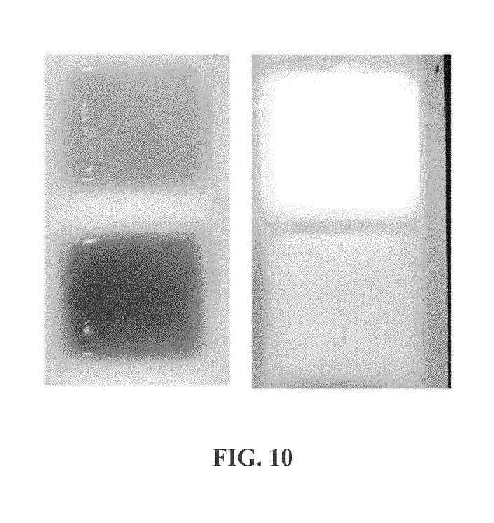

FIG. 10 shows ice pops containing NpF2164g5 (top) and NpR6012g4 (bottom) under white light (left) or ultraviolet transillumination (right). White light produces a mix of the two photostates in NpR6012g4, and ultraviolet light induces only weak red fluorescence. NpF2164g5 instead exhibits intense fluorescence upon transillumination, saturating the response of the digital camera under these conditions.

We have focused on proteins that were readily available at the time proof-of-concept experiments were carried out, but other CBCRs with other colors are also available. Moreover, ongoing surveys of CBCR diversity may isolate additional colors. CBCRs not explicitly presented herein can nevertheless be used as food coloring agents, as can related photosensors such as phytochromes.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

### V. REFERENCES

Aravind and Ponting (1997) Tr. Biochem. Sci. 22: 458-459.
Cornejo et al. (1992) J. Biol. Chem. 267: 14790-14798.
Essen et al. (2008) Proc. Nat. Acad. Sci. USA 105:14709-14714.
Falk (1989) The Chemistry of Linear Oligopyrroles and Bile Pigments, Springer-Verlag, Vienna (pp. 355-399).
Gambetta & Lagarias (2001) Proc. Nat. Acad. Sci. USA 98:10566-10571.
Greene and Wuts (2007) Protective Groups in Organic Synthesis, 4th Ed., Wiley-Interscience, New York.
Hirose et al. (2008) Proc. Nat. Acad. Sci. USA 105:9528-9533.
Hirose et al. (2013) Proc. Nat. Acad. Sci. USA 110:4974-4979.
Hurley et al. (2000) EMBO J., 19: 5288-5299.
Ikeuchi & Ishizuka (2008) Photochem. Photobiol. Sci. 7:1159-1167.
Narikawa et al. (2013) Proc. Nat. Acad. Sci. USA 110: 918-923.
Rockwell et al. (2008) Biochemistry 47:7304-7316.
Rockwell & Lagarias (2010) ChemPhysChem 11:1172-1180.
Rockwell et al. (2011) Proc. Nat. Acad. Sci. USA 108:11854-11859.
Rockwell et al. (2012) Biochemistry 51:1449-1463.
Rockwell et al. (2012) Biochemistry 51:9667-9677.
Rockwell et al. (2014) Proc. Nat. Acad. Sci. USA 111:3871-3876.
Rockwell et al., (2015) Photochem. Photobiol. Sci. 14:258-269.
Rockwell et al. (2015). Photochem. Photobiol. Sci. 14:929-941.
Sambrook et al. (1989) Molecular Cloning: A Laboratory Manual, Cold Spring Harbor Laboratory Press, New York, N.Y., 2nd ed.
Terry et al. (1993) J. Biol. Chem. 268:26099-26106.
Wagner et al. (2005) Nature 438:325-331.
Weller et al. (1980) Chem. Ber. 113:1603-1611.
Yang et al. (2008) Proc. Nat. Acad. Sci. USA 105:14715-14720.
Yoshihara et al. (2004) Plant Cell Physiol. 45:1729-1737.
Yoshihara et al. (2006) Biochemistry 45:3775-3784

### VI. EXEMPLARY EMBODIMENTS

Exemplary embodiments provided in accordance with the presently disclosed subject matter include, but are not limited to, the claims and the following embodiments:
1. A food composition comprising a food and one or more recombinant cyanobacteriochromes (CBCRs).
2. A food composition comprising a food and one or more isolated or purified CBCRs.
3. The food composition of embodiment 1 or 2, wherein the one or more CBCRs are selected from the group consisting of NpF2164g5, NpR6012g4, NpF2164g3, NpAF142g1, NpAF142g2, NpAF142g3, NpF0020, NpF1000, NpF1183, NpF1883g2, NpF1883g3, NpF1883g4, NpF2164g2, NpF2164g4, NpF2164g6, NpF2164g7, NpF2854g1, NpF2854g2, NpF2854g3, NpF4973, NpF6001, NpF6362, NpR1060, NpR1597g1, NpR1597g2, NpR1597g4, NpR1759, NpR2903, NpR3691, NpR3784, NpR3797, NpR4776g1, NpR4776g2, NpR4776g3, NpR5113g1, NpR5113g2, NpR5113g3, NpR5313g1, NpR5313g2, NpR6012g2, NpR6012g3, Synechocystis Cph1, Synechocystis Cph2, CparGPS1, GwitGPS1, EsPHL1, DtenPHY1, NpyrPHY1, PcolPHY1, TastPHY1, Ava_3771, Anacy_2551g3, Anacy_3174g6, Anacy_4718g3, Apl_4973, WP_016873240, WP_016878855, AFZ15460g3, Cyan7822_4053g2, Cyan8802_2776g1, UYIDRAFT_04680, WP_016871037, M595_0799, Mic7113_2205, Mic7113_2408, LYNGBM3L_56870g6, Nos7524_4790, Fdi_DRAFT46470, Pleur7313DRAFT_05530, WP_033374293, Sta7437_1656, Syn7502_01757, Oscil6304_1286, Oscil6304_4336g2, Oscil6304_4065g2, Oscil6304_4080, Oscil6304_4203, Oscil6304_2705, Oscil6304_3021, Oscil6304_4174, RcaE, Tlr0924, and combinations thereof.
4. The food composition of embodiment 1 or 2, wherein the one or more CBCRs are selected from the group consisting of NpF2164g5, NpR6012g4, NpF2164g3, NpR4776g3, Tlr0924, NpR5113g2, NpF1883g3, NpF2164g6, Anacy_2551g3, Anacy_3174g6, and combinations thereof.
5. The food composition of embodiment 1 or 2, wherein the one or more CBCRs comprise one or more truncated CBCR domains.
6. The food composition of any one of embodiments 1 to 5, wherein each of the one or more CBCRs independently has a length of less than about 230 amino acids.
7. The food composition of any one of embodiments 1 to 6, wherein at least one of the one or more CBCRs exhibits a color change in response to light comprising a photoconversion wavelength.
8. The food composition of any one of embodiments 1 to 7, wherein at least one of the one or more CBCRs exhibits fluorescence in response to light comprising a fluorescence excitation wavelength.
9. The food composition of any one of embodiments 1 to 8, wherein at least one of the one or more CBCRs exhibits a color change in response to a pH having a protochromic pH value.
10. The food composition of any one of embodiments 1 to 9, wherein the composition comprises a first CBCR having a light absorption at a first wavelength, and a second CBCR having a light absorption at a second wavelength, wherein the first wavelength is different from the second wavelength.
11. The food composition of embodiment 10, further comprising a protein, wherein the first and second CBCRs are each domains of the protein.
12. The food composition of any one of embodiments 1 to 11, wherein the food is a beverage.
13. The food composition of any one of embodiments 1 to 11, wherein the food is not a beverage.
14. The food composition of embodiment 13, wherein the food is an ice pop, a frosting, a glaze, a shell, or a coating.
15. A food coloring composition comprising one or more recombinant CBCRs.
16. A food coloring composition comprising one or more isolated or purified CBCRs.
17. The food coloring composition of embodiment 15 or 16, wherein the one or more CBCRs are selected from the group consisting of NpF2164g5, NpR6012g4, NpF2164g3, NpAF142g1, NpAF142g2, NpAF142g3, NpF0020, NpF1000, NpF1183, NpF1883g2, NpF1883g3, NpF1883g4, NpF2164g2, NpF2164g4, NpF2164g6, NpF2164g7, NpF2854g1, NpF2854g2, NpF2854g3, NpF4973, NpF6001, NpF6362, NpR1060, NpR1597g1, NpR1597g2, NpR1597g4, NpR1759, NpR2903, NpR3691, NpR3784, NpR3797, NpR4776g1, NpR4776g2, NpR4776g3, NpR5113g1, NpR5113g2, NpR5113g3, NpR5313g1, NpR5313g2, NpR6012g2, NpR6012g3, Synechocystis Cph1, Synechocystis Cph2, CparGPS1, GwitGPS1, EsPHL1, DtenPHY1, NpyrPHY1, PcolPHY1, TastPHY1, Ava_3771, Anacy_2551g3, Anacy_3174g6, Anacy_4718g3, Apl_4973, WP_016873240, WP_016878855, AFZ15460g3, Cyan7822_4053g2, Cyan8802_2776g1, UYIDRAFT_04680, WP_016871037, M595_0799, Mic7113_2205, Mic7113_2408, LYNGBM3L_56870g6, Nos7524_4790, Fdi_DRAFT46470, Pleur7313DRAFT_05530, WP_033374293, Sta7437_1656, Syn7502_01757, Oscil6304_1286, Oscil6304_4336g2, Oscil6304_4065g2, Oscil6304_4080, Oscil6304_4203, Oscil6304_2705, Oscil6304_3021, Oscil6304_4174, RcaE, Tlr0924, and combinations thereof.
18. The food coloring composition of embodiment 15 or 16, wherein the one or more cyanobacteriochromes are selected from the group consisting of NpF2164g5, NpR6012g4, NpF2164g3, NpR4776g3, Tlr0924, NpR5113g2, NpF1883g3, NpF2164g6, Anacy_2551g3, Anacy_3174g6, and combinations thereof.
19. The food coloring composition of embodiment 15 or 16, wherein the one or more CBCRs comprise one or more truncated CBCR domains.
20. The food coloring composition of any one of embodiments 15 to 19, wherein each of the one or more CBCRs independently has a length of less than about 230 amino acids.
21. The food coloring composition of any one of embodiments 15 to 20, wherein at least one of the one or more CBCRs exhibits a color change in response to light comprising a photoconversion wavelength.
22. The food coloring composition of any one of embodiments 15 to 21, wherein at least one of the one or more CBCRs exhibits fluorescence in response to light comprising a fluorescence excitation wavelength.
23. The food coloring composition of any one of embodiments 15 to 22, wherein at least one of the one or more CBCRs exhibits a color change in response to a pH having a protochromic pH value.
24. The food coloring composition of any one of embodiments 15 to 23, wherein the composition comprises a first CBCR having a light absorption at a first wavelength, and a second CBCR having a light absorption at a second wavelength, wherein the first wavelength is different from the second wavelength.
25. The food coloring composition of embodiment 24, further comprising a protein, wherein the first and second CBCRs are each domains of the protein.
26. A method of coloring a food, the method comprising adding a food coloring composition of any one of embodiments 15 to 25 to a food.
27. The method of embodiment 26, wherein the food is a beverage.
28. The method of embodiment 26, wherein the food is not a beverage.
29. The method of embodiment 28, wherein the food is an ice pop, a frosting, a glaze, a shell, or a coating.
30. The method of any one of embodiments 26 to 29, further comprising subsequently illuminating the food with a light comprising a photoconversion wavelength, wherein at least one of the one or more CBCRs exhibits a color change in response to light having the photoconversion wavelength.
31. The method of any one of embodiments 26 to 30, further comprising subsequently illuminating the food with a light having a photoconversion intensity, wherein at least one of the one or more CBCRs exhibits a color change in response to light having the photoconversion intensity.
32. The method of any one of embodiments claims 26 to 31, further comprising subsequently adjusting the pH of the food to a protochromic pH value, wherein at least one of the one or more CBCRs exhibits a color change in response to a pH having the protochromic pH value.

INFORMAL SEQUENCE LISTING
The following Informal Sequence Listing provides exemplary amino
acid sequences of cyanobacteriochromes (CBCRs).

NpR6012g4 (SEQ ID NO: 1)
EKAVTKISNRIRQSSDVEEIFKTTTQEVRQLLRCDRVAVYRFNPNWTGEFVAESVAH
TWVKLVGPDIKTVWEDTHLQETQGGRYAQGENFVVNDIYQVGHSPCHIEILEQFEV
KAYVIVPVFAGEQLWGLLAAYQNSGTRDWDESEVTLLARIGNQLGLALQQTEYLQQ
VQGQSAK
NpR4776g3 (SEQ ID NO: 2)
QQRVLFEVVAKVRKSLDLDAIFQTTTQEICKSLQADRVAVFQFQADWSGEYIAEFVG
DGWVKLVGSNTKTVWQDSYLQETQGGRYRHNETFAVDDIYQVGHSQCHVAVLEQI
QARAYAIAPIFIGQQLWGLLAAYQNSAPRHWEASEIKFITQIANQLGVALQQAQLHN
QTKEQ
Tlr0924 (SEQ ID NO: 3)
EREMIVSTIIQDIRQSIRLEEILQRAVNSIQQLLLSDRVLIYRFLGDGSGIVAVEATTLPQ
YSILGQVIHDPCFTKETARRFLEGRTLSISDVNQAQLQDCYRELLTRLQVQANLVVPL
LQGQHLWGLLIAHHCRSPRLWQREELFLLQRIAEPLTVALQQAEMYEALEQ
NpR5113g1 (SEQ ID NO: 4)
PNSNFAENQLLLSSAQLQEQLDQQKALASVIVRIRESLDLETIFQTTATEVRLLLNAD
RVAVFQFDLEKDWEGEFVSENVAPGWTSALKVKVYDHCFGEQFASSYQQGRVQAV
ADIYNAGLSDCHAAILSKFQVKANLVVPLSRQKELWGLLCIHQCSEARNWKESEIEFI
RQIADHLTVAIQQAKHLHEVQ
NpR5113g2 (SEQ ID NO: 5)
RDQVIAQIVDKIRSPLDIETIFKTTTQEIRKLLQADRVAIFSFNADWSGNFVAESFAEG
WTPLVGIQPTIFDTYLQQTQGGRYVDNETFTVNDIYQAGLTDCHVTLLEQFQAKAFA
TAPILQGDKLWGVIAAYQNVSPRQWQSYEVESLTKIGTQIGVALRHYKLLAH
NpF1883g3 (SEQ ID NO: 6)
MQSQLLADTIGSIRQSLNEEDVLKTTVEEVRKVLSTDRVMIYSFNANWSGTVIAESV
VLTYPKVLRAEIEDPCFGQGYVKEYQSGRVLAINNIYEVGLADCHINLLESFGVKAN
LVAPILKDEQLFGLLIAHQCSRPRDWKQSEIDLFAQIAMQVGFALDHARLLQR
NpF2164g3 (SEQ ID NO: 7)
ERERTVFGHEKIGQATDLRTLFRTATQELRRFLKCDRVVVYQFHPDWSGEVIAESVG
AGWSSLVEMQEQDGILKTGLISSERCNIKDYGSPVKADADTYLKETQGGMYAKGTR
FRKVNDISAAGFSSCYLESLEKFQAKAYIIVPVFRAGNLWGLLAAYQNSEVRQWTEE
EAGALLQIAEPLSIALQQAEYIEQLRLKNLE
NpF2164g5 (SEQ ID NO: 8)
EEQVLTAMVEKIRQAQDMDTIFLNVLPSLRKQLQCDRLAVFRFHPDWSVEFVAESV
KDKWLSLADSDIKTIWMDEHLQETQGGRYRNHETFVVNDIYTVGHVQCYLEILEKIQ
AKAYAIAPIFIGNKLWGFIGAYQNTGPREWKSKDVQLLRKVAVQMGIGLQQVKYIE
Q
NpF2164g6 (SEQ ID NO: 9)
ERALTRLSEKIRQVQELDTIFRNALPELRSHLECDRLAVYRFNPDWGGEFIAESVSRE
WVALVGPEIRTIWEDEHLQQTQGGRYRNNETFVINDVYTAGHAQCHLKILEQFQIRA
YIITPIIAGNKLWGLLGAYQNSGSRQWQENEVNLVAKIGTQFGVAVQQSQYLQQ
Cyan7822_4053g2 (SEQ ID NO: 10)
EQIRQKLRQQQAIAAIVQQVRQSLNIEEILNTITQDVRALFDCDRVIIFRLYSDGGSRII
EESVSTEFLPLKYCHWDDETWSQDILNLYWQGQPRIVPDVMNDIYTECLHEYSREGQ
IQSKIVAPILLDLKEKENHRWVASTNSHKLWGILVVHACREKRVWQNSEAQLLQQIA
NQLAIAIQQASLFEQ
Sta7437_1656 (SEQ ID NO: 11)
ERLITTITQNIRQSLDLKKILATTVIEVQQTLNAERVLIFRMNPDGSGQVIEEAVVPKY
PVTDQMRWEDEHFPEDCYEYYRQGIPRIVPDVATDEWAKCLVEFMQEVGVKSKVV
APIVQVYEKSSTNAKVWGLLIVHACSHYRQWQESEVDFLQRIGNQLAIAINQANLYQ
QLQAE
Anacy_2551g3 (SEQ ID NO: 12)
EDAISLQLQRQKIIQDITQQIRSTLNVNHILATVTQQVKELMQVERVIIFRLFPNGRSQI
VEEVVSSEYAALKNYHWEDEKWSQEILDCYWQGKPRIVPDVINDIWTSCLVEYTTQ
GNIQSKIVAPILQELGENETGRWVSSEHKQKLWGVLVVHACSTKRVWEEDEAQLLQ
QIANQLAIAIQQAALFEQLQ
Anacy_4718g3 (SEQ ID NO: 13)
ELAISHQLQQQRTLGKIVQKIRDSLDIKDILATVTQEIKNSLNCDRVIVFRLFADGESQI
VEEAVSGELVSLKNRHWDNEVWSQEILDYYWQGQPRIVTDVMEDIWTDCLVEYSIE
GQVQSKIVAPILQEAQDGEKNRWVASGENNKLWGVLVVHACSEKRIWKDCEAQLL
QQIANQLAIAIQQANLFEKLQQ
WP_016871037 (SEQ ID NO: 14)
ERLLITITQHIRQSLDLEQILRTTVVEVQRTLQTDRVLIFRLNQDGSGQIIEEAVVPEYP
MTYQMRWVDECFPDDCYEYYRQGNPRILPDVAKDEWGACLVEFMQQIGVKSKVV
APIIQTLEDSSTRVWGLLIVHACSHYRQWQASEAEFLQQISNQLAIAIHQADLYYQLQ
IE
NpR4776g1 (SEQ ID NO: 15)
MTSNPEQNFLYSQEESLLRRITNRIRRSLELEEIITVTTAEVRSLLKTDRVMIYKFHAD
GNGQVIAESIYNNRLPSLLGLNFPADDIPLSARELFLKLRVRSVVNVDTQEIGQIHLRD
LDNGETISEEIRYRSVDSCHIEYLTAMGVKSSVVAPILYQDQLWGLLVSHNSEARLIS
EYELEAVQMVVEQLSVAIAQSSLLTQVRK
WP_016878855 (SEQ ID NO: 16)
QQERLLGTITQHIRQSLNLEEILATTVIEVQQTLQADRALIFQLNQDGSGQIIQEAVIPD
YPVTNQMRWLDECFPDECYEYYCQGNARIVPDVAKDDWGACLVEFMQEVGVKSK
VVAPIVQSFEGSSNKVWGLLIVHACSHYRQWQASEVEFLQQLCNQLAIAIHQANLYH
Q
WP_033374293 (SEQ ID NO: 17)
EAMIAQQLRQRTVLGQIVQQIRESLNLQEILAITTQRVREILQGDRVIVFRFCDLGRTC
IFEEAVAEDLPSLKYMNWEDEQWSSEILQFYWQGQPRIVPDVMNDPLTPCLLDYSRQ
GQIQSKIVAPILQEIHNGERGNGEIDPWTDPESGNKLWGLLVIHACHEKRIWQESEAE
LLQQIANQLAIAIRQSRLFEQLQE
WP_036801164 (SEQ ID NO: 18)
RRRQAEIFLWKQSERERVINQITQQIRQSLNLDEVLSTTVKEVRKFLDCDRVLIYRIRE
DGTGSTITETVLSPYPAILGQTFPEEVFPTEYHQAYIQGKSRTITNIEQDDVEECLADFV
KQFGVQAKLVVPIIQETRDISLSEEKQTTAPYLWGLLIAHQCSQTRQWQPLEVQLMQ
QLATQVAIAIQQSELYTQLQQ
O6304_4080 (SEQ ID NO: 19)
QAEEAMRQQVERERLLGAIAQRIRQSLDLTTILSTTVEEVQQVLGCDRVLVYRVWA
DGTGSAIAEAVKPGWLKVLNRVFPEEVFPPENYQRYIEGRICALVDRDSGQTLPCLV
EFMKSIEVRAKLVVPIVEKDTLWGLLIAHQCSGPRQWQGWEIDLLASLATQLAIAIQ
QSELYERLQVELSDR
UYIDRAFT_04680 (SEQ ID NO: 20)
EQSLQLQAEQERLLSTITQHIRQSLDLEQILRTTVVEVQQTLQTDRVLIFRLNQDGSGQ
IIEEAVVPEYPMTYQMRWVDECFPDDCYEYYRQGNPRILPDVAKDEWAACLVEFMQ
QIGVKSKVVAPIVQTLEDSSTKVWGLLIVHACSDYRQWEASEAEFLQQISDQLAIAIH
QADLYQQLQIELAER
NpF3797 (SEQ ID NO: 21)
ERERLVNQIAQHIRESLELDEVLTTTVAEVREFLQADRVLIYRLWEDGTGSAITETVL
PEYTKILGETFPEEVFPREYHQAYSLGKTRAIANVEQADVESCLADFVKQFGVKAKL
VVPILQENREADKQRDSKTPASPPYLWGLLIAHQCDRTRVWESWEIELMKQLATQV
AIAIQQSELYK
RcaE (SEQ ID NO: 22)
QRVELFSEVTLKIRQSLQLKEILHTTVTEVQRILQADRVLIYHVLPDGTGKTISESVLP
DYPTLMDLEFPQEVFPQEYQQLYAQGRVRAIADVHDPTAGLAECLVEFVDQFHIKA
KLIVPIVQNLNANSQNQLWGLLIAHQCDSVRQWVDFELELMQQLADQISIALSQAQL
LGRLEE
WP016873240 (SEQ ID NO: 23)
KREQLLRGITQRIRQSLDLEQILATTVNEVLQTLQSDRALIFRLHGNGTGQVIQEAVRP
EYPVTEQMLFPDECFPQECYEYYCQGQPRIVSDVFAEDFSSCLVEFMQKIGVKSKIVA
PIVQTTENSSTKVWGLLIVHACSQHRQWQQSEADFLQQISNQLAIAIQQSQLYQQTR
QQ
Nos7524_4790 (SEQ ID NO: 24)
RERLLRNITQHIRQSLDLESILSAAVNEIRQTFQAERALIFRLMPNGSGVVLEESVVPD
YPSIKSRLWQDECSPHELYQFYRDGNVRIVTDTQNDLWGECLAEFMAEAQVQSKIV
APIAQKLDNQPPRVWGLISIHACGKKRQWQTDEANLLQQIASQLSVAIQQADLYQQV
QTE
Syn7502_01757 (SEQ ID NO: 25)
ERQNFITKITQQIRQSLDLEEVLTAAVNEVSSLLNCDRIIIFQLFPNGASQVVKEIVKPP
YPSIIGMNWQDEHFSHEGFEYYINSNPRTVNDVFEDNVFADCLKEFMSQAQIKSKIV
APIIQHIQSCSLEPYNRWRRDNSQLWGLLIVHSCGEYRQWDQEEADLLQQVANQLAI
AIQQADLYQQVQQE
NpF1000 (SEQ ID NO: 26)
KQISQERLLNQVTTQIRKSLDLPVIMATAITQVREFLELDRLVIYKFEASKVKTQEYQS
STDEDNGKGSTSISVNNQSLLEDYQHRGYIVYEARATDAITCVLNYTEKNCFMRTSQ
CWEKYRQGFTLAVDDVEKTYALEECLLNFLRESQVRAKLAAPIILENKLWGLLIAHQ
CNEPRQWIDSEKNLLIAIAEQLAIAIYQAELMQTLTQEKQTLEH
O6304_4203 (SEQ ID NO: 27)
REMLLSRISAKIRSTFDLPTILKLGVQGLRQALDTDRALAFQFLADGSGVCIAEDVQA
PYPSIRGQWFPADCMPLTHRDAYRNGHLGSVPDIQNAALSDCYREMLNQFQVRSFM
GVAIGRNQEGSIIPDSLSFKELQNSTGEISEIDSNVLKSPSVSSLWGLLVVHHCHGPRQ
WTMDEKQLVQAVATQMAIAIEQANLVQQLRTYARE
O6304_4336g2 (SEQ ID NO: 28)
ELQQSLKFTRILKQITDQIRSTLDVSTILQTIAQRVRNLLNTDRVVIYQFDDMGNGEVT
VEDIRESWQSVRGVRCPGCIPPDVSNLYLKGRIRAINNVATAELSACHREFLDSIQVK
ANLIVPICMGSDLWGFLIAHECEQPRLWQEQEIDLLQQLADQAAIAIAQAELYQQTQ
NpR5313g2 (SEQ ID NO: 29)
TKVIKQITEQIRRTLDLQTTLQTIVSEVRSLLNSDRVVIFQLNSKSVIVEEMNGNWQSV
LGVNAPPECFPNEHRDLYSQGRVRAINNVSTDSLSDCHREFLQSLQIQANLTVPINIGI
ELWGLLIAHECNTPRNWQDVEIDLLQQLGDQAAIAIQQAQLYEQTCKAETEARNK
O6304_3021 (SEQ ID NO: 30)
ERERVMSAMRDRIRSSLNLKEILNTTVAEVRQFLNTDRVITYRFHPDWSGKVVVESC
GTRAREILNLTILDPCFGETHAQLYKKGRLKATPDIYTEPISPCHRNLLAQFQVKANL
VVPILLNEDFSHNSEEELLENSVGDNNQLWGLLIAHHCQGPRQWQNWETEFLQELA
TQVAIAIQQSTLFEQLQ
NpF4973 (SEQ ID NO: 31)
MQIHPHSEFNHSRDRREQGLQNLLDRLVKSIQRDELVRQTTNQLRESLQVDRVVLYY
FYSQWQGQVTFESLSSKEFSILSSTGPDDCFNNEYAALYLAGRVRAIANIELEPIQPCH
RDFLRNLQVRANLVVPIVIPRGLWGLLVAHHCQGPHDWSSSDIEMMQTGAQTLATD
RNILES
Apl_ORF1 (SEQ ID NO: 32)
MNFPNIFGDSTNSNPNPNSDPRFQQFVERLQQVEDRDRLIQETLNGLRKHLKSDRIVL
YYFYKQWRGQVTFESLRYIDYSIYGSTGADDCFNDQYARLYLEGRISAISDIDEADID
SCHRDFLKSIRVKANLAVPVVKNQQLWGLLIAHYCQTSHSWVNAEIDTMKQAAKLL
SESPFLD
O6304_2705 (SEQ ID NO: 33)
MSDWSLRQVADRLAKTLNQDLLIRQTTLELREILNVNRILLYYFYSPWKGQVTFESV
SAPQFSIIGQSGPDQCFGDEYAALYLQGRVRAISNIETEPINECHREFLRTLQVKANLV
VPVLSPIGLWGLLVAHHCEDTRLWSLDDIKRMKNAAETLGTAATIRDS
NpF1883g2 (SEQ ID NO: 34)
AQIFIEITRSIRESLNEEDVLKTTVEEVRKVLSTDRVMVYTFNANWSGTVIAESVIPGY
PKVLRSEIQDPCFGQGYVEKYQSGRVATTNNIYQAGLADCHISLLESFGVKANLVAPI
LKDEQLFGLLIAHQCSRPRDWQQPEIDLFAQIAIQIGFALDHARLLQR
NpF1883g4 (SEQ ID NO: 35)
RSQLLVDITRSIHQSLKEEDVLKTTVEEVRKALSTDRVLVYSFYANWFGIIIAESVVPG
YPKVLRSKIHDSCFTQGYIEKYQSGRVVAINNIYESGLADCHIKLLESFAVKANLVAPI
IKDDQLFGLLIAHQCSGPRDWQQPEIDLFTQIAMQVGFTLDYARLLQAYQATEASSN
NpR1597g1B (SEQ ID NO: 36)
MENSSTTQPIEPNSEQQESHSAVTSSLRQNEQQKALSGVIARIRESLDIETIFKITVTEV
RQLLKTDRVGVFRLYPDLAWEGEFIYEDVATEWDSALAAKLCDHCFSEEFASLYQQ
GRIRAIADIYQVNASDCYIQILERFQVRANITAPLIKGKNLWGLLCIHQCSSSRQWEAS
EIEFVQLIAEHLGVALQQADYLEQVKLQSAQLAQAKAR
NpR5113g3 (SEQ ID NO: 37)
EQQKTLTSVITRIRESLDLDTIFQTTVTEARKMLQADRVAVFRFDPQKDWEGEFISED
VVPECNSVIAEKIYDYCFGENFASLYAQGRVNAIADIYQAKFPGCYIQILEKFQVRAN
LVAPLLKKGELWGLLCIHQCTTFRHWQPSEIEFTSQIAEQLGVALKQDSYLKQVQMQ
VVQLAEATER
NpAF142g3 (SEQ ID NO: 38)
QQTLASVITKIRESLDLNAIFETTTQELRRVLNCDRVVIFRFYSESNYDGGEVIAEDVA
ERFLSTLTAKVYDRCLGQEHIEKFCQGYVHAVTDIYNSELNECYVSMLSRFQVRANL
VIPMLKQGQLWGLLCIHQCSRPRAWQDSEIEFVRQIAAQLGVALQHVVLLNQ
NpR2903 (SEQ ID NO: 39)
LQNLRSQLFANITLKIRQSLQIDEIMQTSVKEVQKLLLADRVLILRLQPNGSFIAVQEAI
VPGLPVVLGQQITDPCFRDDYIEQYRQGQINVINDIKEADIQPCHVELLQRFAVKANL
VVPIFLKNQLWGLLIAHQCAHPRQWTSWEIELLRQLADQIGIASTQSLILEQETRQR
NpF6362 (SEQ ID NO: 40)
KRQNLRSQLFAEITLKIRESLQIDEILQTTVTEVQKLLQADRVLIFRLEADGSGTVVQE
AVLPGWPVILRKNLFDPCFKEEYIERYRQGRVSAMEDIETAHIQPCHREFLQQFAVRA
NLIVPILVRDRIWGLLLAHHCAAPRKWNNFETELLKQLANQIGIALSQAELLEKETQQ
S
NpR1060 (SEQ ID NO: 41)
ERIVAAVTQRIRQSLELDEILQTTAAEVRQLFEVDRVIIYQFESDWSGLVAVESLAEEC
MSILGFHVMDTCFKSTRAIYFQQGNTRAIEDVETAGLSPCHLDLLRSLQIRANLVVPIL
QKERLWGLLIAHQCRNARQWQQSEINLFNQLAGQAAIAIQQSELYHQLQQANQE
NpF6001 (SEQ ID NO: 42)
EREQLMRTVAQRIRQSLNLQDILNATVQEVRDLLGVDRVVVYQFDSEMIGTIMAESV
EPGWRVSLGVEIYDTCFQTGKGAEYYQVNKRAIANIYEAGLTDCHIRLLEQFEVKAN
LVVPILLEVSSQTPGSHLWGLLIAHQCSGFREWEKNQLDLLDQVTVQLAIAIQQSSILE
QAQTELVQR
Mic7113_2205 (SEQ ID NO: 43)
QREQLLGEMQGRIRQSLDLEEILSTTVSEVRQFLQTDRVLIYRFFDDWSGVVAVESVS
TDELSILNTTISDPCFGEAYVERYQQGRIMVIEDIYTAGMTPCHVDFLASIQVRANLV
VPILQKERLWGLLVAHHCYEPRQWQQWEVDFLEALSTQIAIAIQQGQLHQRLYS
Cyan8802_2776g1 (SEQ ID NO: 44)
ELQTQDFTLSVTDGENTLKPLENWFSRALAKARQLLGADRLLIYRFAPDGRGTIDYE
SLGSGVSSTLNQQIGDACIPAELLEAYRVDRVAMINNLSEVTLDPDHLELLTRLGVQA
NLVIPILNQGNLFGLLIAHQCFSPRQWEERDINFLKQLSTQFQVILNRLSLSHQQ
NpR3784 (SEQ ID NO: 45)
MGNNDEFNSMCHLTECQQAEEALQQQIQWQRLVMAIAQRIRQSLNLDKVLNTTVTE
VRQFLQVDRVFMYQFEPDYSGVVVVESVDDRWIAILNTQVQDTYFMETRGEEYSHG
RIQAIADIYTAGLTECHRDLLTQFQVRANLAVPILQGKKLWGLLVANQCAAPRQWQ
TWEIDFLKQLAVQVGIAIQQSQLYEQVHTELAER
Ava_3771 (SEQ ID NO: 46)
NHDEYNQESLKNVNKQAQEALQQQIERQRLVMAIAVRIRESLRLNEVLNTTVAEVR
QFLQADRVFIYRFAPNYSGCVVVESVGDNWRAVLNAQVEDTYFMETHGEKYRQGG
LQAIADIYAEDISECHRDLLIQFQVKANLVVPILQGEKLWGLLVTNQCGTSRHWQSW
EIDLLQQLATQVGIAIQQSELYEQVRAE
Mic7113_2408 (SEQ ID NO: 47)
CKASLEESCQHSEEALRQQFLWEQLLGAIAQRIHQSLNLDEILNTTVDEVRQVLACD
RVIIFRLHPDGSGVIVVESVDSGWRPISGISINDHHFAQAYIKLYKQGRVQAVEDIYTA
DLTPCHRDLLAQLQVRANLVVPIVHEEQLWGLLVAQQCSKPRMWKSLEIDLLSSLA
TQAAIAIQQSELYQQAQTEIAQR
WP_023064634 (SEQ ID NO: 48)
QRQKILFEIVNRIRQSLDLQTIFDTTTQEVRRFIKSDRVVVYRFNSDWSGYFVAESFSS
EWKPLIDLIPNIADTCLQETEGGRFRRGEILAIEDIYDAGYSDCQIKLLEQFSARAYIVV
PILQGENLWGLLAAYQNTATRRWTDEELQLLSQISIQLGVALQQAELLAETQ
Anacy_3174g6 (SEQ ID NO: 49)
EQEKAISKIVNRIRQSLDVEEIFKTTTQEVRLLLKCDRAAVYQFTSDWGGEFVAESVV
SGWVKLVGPGIKTILNDPCLQNNEGGRYKKGDTLIVNDIYTATLDPCYIEHEKYEAR
AYIIAPILFGEKLWGLLAAYQNSGTREWEESEINLLSRISDQLGLALQQTEYLQQVQ
NpF2854g3 (SEQ ID NO: 50)
RQRALTEVVGKIRSSLNIDLILKTTCQEVCKLLRVERVGVYRFNEDWSGEFVSNFGM
VEAQWDSINPFGKNLVWEDTHLQETKGGRYRNNENFAVEDIYQAGHSRCHLDILEQ
FKIRAYALTPIFVGRNLWGLLAAYQHSAPRQWDSVEVEFLGQVASQLGVALQSSQM
VTQIQTRADE
NpR1597g4 (SEQ ID NO: 51)
REKALAATVEKIRQSLDIGTIFATSTEEVRRLLEVDRVTIYRFRADWSGEFVAESLAQ
GWTPVREIVPVVADDYLQETQGRNFANGKSIVIKDIYSANYSICHIALLELMQARAY
MIVPIFQGEKLWGLLAAYQNIKPRDWQEDEVDLVMQIGTQLGVGLQQAELLE
NpF2854g1B (SEQ ID NO: 52)
ITNSPKTSNLLDLQECHKQLQLTVQYQKILARILAKIRASVNIESICSTSCQDICRQLKI
ERVAIYRFNTDWSGSFINRFGFAEHPWDALTAFGQDLVWQDSHLQETQGGRYRKNE
PFAVADIYDAGHARCHIEVLEQFQIRAYAIAPIFIGAKLWGMLAAYQHSAPRQWWPH
EVEFLAQAASHLGVAMQQAETLEQTKQR
NpF2854g2 (SEQ ID NO: 53)
RQRALTEVVGNIRSSVNPEIILDTACQELCKLLKLERAAIYHFNEDWSGEFVSQFGML
EPQWHRIHPFGKNLVWQDTHLQETKGGRYRNNESFVVNDIYQAGHSRCHIDILEQFK
IRAYTLAPIFIGPKLWGLIAAYQHSGPRQWANYEVEFLGQVGAQLGVAIQQAENLAQ
SKQ
NpR6012g2 (SEQ ID NO: 54)
KKSVAKVIDKILRASNVDKIFKTTTQEVRQLLKCDRVAVYRFKPDWSGEFVAESVGN
GWVKMVSPDFYMVWEDSHLQDTQGGRYAKGESFVAKDIYKMGHAQCHIDILEQYE
MKAYIIAPIFAGEKLWGLLAAYQNSGPRDWQPWEESFVTQIGLQFGVAISQGEYLEQ
MHKKSE
NpR6012g3 (SEQ ID NO: 55)
EKAFTKIVGRIRQSLDVDSVFKTTTQELRQSLRCDRVAVYRFNPDWGGEFVAESVGT
GWTKLVGPDIKTVLDDTYLQETKGGRYVRGENFVVNDIYQVGLAPCHIEILEQFEAK
AYIIVPIFFGDKLWGLLAAYQNSRPREWETWEVTFLVQTSLQFSLAKSQIDYLELVRL
KSEK
NpAF142g2 (SEQ ID NO: 56)
ERQQALARIIANMRQSLDVTTIFRTTTQEVCQLLECDRLSVYRFNADWGGEFVGDYE
TANPRWGRSIKLGVGMVWDDTYLQETQGGRYRNNETFVVDDIHSQGFTQCHIEILE
QFHVQAFMIAPIFVGQELWGLLGAYQHSSTRHWQASEIEFFTQIATQLGVALQQAEY
LEQVRAQTRK
NpR4776g2 (SEQ ID NO: 57)
RTVELQRTAEQEQAVFKVIAEIRESLDTDTIFQTTTKEVCQLIKADRVSVYRFDSNWG
GEFVGDFEAASPYWSNESEIGINTVWNDTYLQDTEGGRYRNNETFAVDDIYKMGFA
KCHIDNLEQFQIHAFVLAPIFVGQKLWGLLATYQHTGPRQWKASEVNFLSQIAAQM
GVALQQAELLTQTR
NpF2164g4 (SEQ ID NO: 58)
ENAIARMADRMRNSQQLETIYRTTLAELRQLFKCDRLAIYRFNSDWSGEFIAESVGS
DWVPLVGPDIKTVWEDEHLQQTQGGRYRNNETLAVNDIYTVGYAQCHIDLLEQFQV
KAYTIAPIFAGNKLWGLLGAYQNSGPRQWNSEEVRLLTKLGIQLGLSVQQVEYIE
NpF2164g7B (SEQ ID NO: 59)
MEREVANARFSYRLPSRLTEMAQSHGNVLEFIQFATHELRQLLKVDRVGVYRFEPD
WSGEFVVESVTGDWPKLVGTSLAKVRDTYLQYNQGGRYVRKESLRVDNIYSVGHD
ECHIQLLEMWGTKAYMISPIFQENRLWGLMGVYQNSASRQWEQSEEDVLNQASVQI
GIALNLADYLTQVRTQEQQ
Moorea_g6 (SEQ ID NO: 60)
ERVVASLSNHLSQSVDVYSVFQSITKELRQVLGADRVVIYRFNSDWSGEFVAESVAP
GWLSLITEQERDDRLKVDTTASERCTVTVLSVNSLSNVDTCLQETQGGDFARGDRFK
QVDDIYAIGFSPCYINTLEQYQARAYLIMPIFQGEKLWGLLAAYQNTGPRQWQDTDV
TLMLQISNSLGLALQRSHYLDQLRAQ
NpR3691 (SEQ ID NO: 61)
ERAIAQVIQRMRQTLDLETIFAATTQELRQVLNCDRVVVYRFNPQWNGEFVSESVGN
GWISLIEEHNNDPNLTEGVLQQGRCLAKILDSGDNQAEDADMQAIQDGIYAQGATFR
CVPDIYNAEFHPSYISLLERFQAKAYIIVPILYGSQIWGLLASYQNSDSRQWKPGEINIV
VQIGNQLGVALQQAQLLAQTQRQSQALQEA
fdiDRAFT46470 (SEQ ID NO: 62)
ERERAIAQVIQRMRQTLDLETIFAATTQELRQVLDCDRVVVYRFHPDWTGEFVSESV
GFGWSSLLEAQKNDPYLTEGALAEGRCIWTMLDSLQQVRDSDLNTIPEIEYTQVANF
RYVADIDKVKFQPNYINFLKRLQAKAYITVPILCGNEVWGLLASYQNSVPRQWKTEE
INIVVQIGNQLGVALQQAQLLAQTQ
NpR1597g2 (SEQ ID NO: 63)
KAAQWQKTIAITIEKIRQSLDLESIFRTSTVELRQLLNADRVAIYRFNPDWSGEFVFES
VAEGWISLIDEQLQRPELSENVSECSAKDLAKPPVADTYLQDTEGGSFSSCEVYRICN
DIYNSGFSDCYIKILEIYQARAYVIIAIYHGQKLWGLLAVYENARTRDWQKDEVYLL
TQVGTQLGVALQQAEFIQQ
NpF2164g2 (SEQ ID NO: 64)
EKDQILVGVANKILQSKDADTIFRVATQEVRQSLKCDRVAIYRFQPDWSGEFVAESV
GSEWVSLLEEQRNNPAIVNNVNYCSVRNLADEGGLAGTTGRSHSADSYIQHTQGKN
FHRGQIYRVTNDIYSAGFSDCYIKILEAYQAKAYIIVAIFQGEKLWGLLAAYQNSQPR
QWKDSEVRLMVQVATLSSITIQQVQYQQQLYHRSE
O6304_4174 (SEQ ID NO: 65)
REKTISLIIQRMRESLNLETIFQATTNALRQAVDCDRVVIYRFNPDWSGALVSESVSTG
WDELLPEQADDPILTQATTDAPGCVLTQMNSSNFLIKDTYLQEQQGGIYRKKQTFCS
VSDIYQAGFSSCYLEFLEQLSARAYVIAPIFSGNTLWGLLGVYQNSGPRDWKEAQVK
IVTQIGNQLGVAVQQAELFAQTQK
O6304_4065g2 (SEQ ID NO: 66)
REKAVASAVQRIRQSLDLGTIFAATTEELQGCLTCDIVAIYRFNPDYSGEFVSEAITSQ
RVSVLQRNRQSLHLSAQPASGENCQMQALINSCLEWQDTHLIQTKGDAFNRDENCL
VVEDIYQRNFSPCYLARLEEFQVRAYIIVPIFCGSKLWGLLAAYESDRPRQWQPSDHK
IVIQIGNQLGVALQQAELLEQTQK
O6304_1286 (SEQ ID NO: 67)
REKALAAAIQTIRRSLDLDTIFAATTQELRTCIDCDRVAIYQFDMNYQGQFVAESING
DWISFLQQSNNPPQLLSNSESEPYCRIEAMLKSPNLWPDSYLMENPAILQDPDKMCL
VVEDVNLANFSSCYQDRLEQFGIRAYVIVSIFCGSKIWGLLAAYQHSGPRKWKASDR
NIVMQIGTQLGVALQQAELLEQK
AFZ15460g3 (SEQ ID NO: 68)
LIKATERERAAAKVIDRLRQSLDIDTIFKTTTRDVRELLKVDRVGVYRFNDDWSGEF
VLESVGAEWTPLLQKSANLEGLQANQSNCHAVQNLGGKRRLADSYLQETEGGRFRE
KGTFAVEDIYKAGFSPCYMEVLEESQVKAYAIVPVYQGEKLWGLLGAYQNSGTRK
WEEAEVALLAQIGDQLGVALQQAEYLQQ
NpF0020 (SEQ ID NO: 69)
MEMNLQFPGINLISLKEAPIHISSQIQPHGVLLVLEEPELKILQVSTNTLKVFGIAPENM
LHKKLEDLLDPFQIERIKTGLSGENLDFINPTKVWVRKKGDEYVVFDAIFHRNIEGFLI
LELEPAITQENIPFLSFYHLARASINQLEKTANLRDFCQVIVQEVRKITEFDRVMLYKF
DDDGHGSVIAEEKLESLEPYLGLHYPESDIPKPARKLFAANSIRIIPDAYSQPVKLFPV
NNPISDRPINLTNSILRSAASCHTEYLHNMGVGASLTISLIKDQKLWGLIACHHQSPKY
VSYELRKACEFLGRVIFAEISAREETEDYYHRINLTHLQSILIEYMSQEENFIDGLVKN
PQHLLDLASAQGAAVCFAGNCTVVGETPREEDLNFLVQWLKNNVEEEVFYTDSLPQ
IYPDAESFKNVASGLLAIPIAKRTYVLWFRPEVIQTVNWGGDPNKAFEVSQSEGNVR
LCPRKSFELWKETVRLTSLPWKDVEVKAALELRKAIVNIVLRQADE
NpF1183 (SEQ ID NO: 70)
MGSQPENTTTQATALTNHDRKPIHIPGSIQPHGILLALSTQLEIVQVSNNTQVYLCKAP
EDLLGRPLSYLLEPQPVEIVKQCLVKKVGSANAFKVLINTLYGEIYFDAIAHRTEEAVI
LELEPTDSEFEVSFLNFHSFASEAIAKMQRTSNLGEFLHLVAQEVQKIISFDRVMVYQ
FDESEAGSVVAEVKREDLSPYLGLHYPATDIPAQARELYTRCFLRFLPDLTAEPVKLV
PTENPTTHQHLDLSYCLLRSFDWCCAEYHQNMGVKALLVISLIQEQKLWGLISCHHQ
TPKYISYEVRKMCEFLGQIVSSELAHKISSSEWDYKVKLKSLQSEFLESTSQADNFIDA
LIKPEIRLLDFVSASGAAVCLDNEINLVGTTPNIDEVRALIEWADTQVTDNLFSTDSLP
KLYPEALIFKDTASGLLLLRISKVRRYYILWFRPEVIQTVHWAGNPQESIKAEGDGSY
TLSPRKSFEQWQETVRLTSLPWKGCELESAIALSNAIVGIVLSKADE
NpR1759 (SEQ ID NO: 71)
MGLVTNSGFRNLKDQFLPERKEALLARMANRIRQSLNLQEILDATVVELRAFLQTDR
TKVYRFDRDGHGHVVAEASAPNRLPSLLGLHYPADDIPPQARALFVKARTRSIVNVS
EQRIILNSLPTPTTTAIGDLTVEEVQEQPLEDILSRPVDPCHVDYLTQMGVQSSLVVPII
YQQELWGLLISHHAEPREITSEDLLVVQLLADQVSLAITQASLLSQVQEQQQRDAIVN
QIATMLHAPLTPEEILHNVLEQAVKASGSSGGRLYLTSPDEALPAHLYTYGNQPAPSE
QEQPHLLENHPLWQEVRKIPAPIGTTLTQKDKPALRVVINLQQEPRLHQUESFQTTPI
RGLIVQPLYYSKEFLGYLTFFRDQISTKNLWPGYEEADERQQRPRQSMAQWQALKE
DKAHPWSIEEMELIQSLNIHLSLAVLQNRLYHRER
NpR5313g1 (SEQ ID NO: 72)
MGTSTDKPNGLQQSLEQESLLHRMIKQIRRSLDLQEILTTTVTEVRSFLRADRVKVYR
FDTSGSGEVIAESIHNERLPSLLGLRFPVHDIPEAAREMFLLAGQRSIVDVANHKIGLS
PLQSTETGKHLQTNIYYRKVDPCHIQYLKAMGVQSSLVVPILDSDPQQSAKPKLWGL
LVSHQSKPRKILKREIKVLQQVADQVAIAIAQSNLLTAALTQQKQEATINRVTTLLHK
LPTIQLQGAIEEVITAFSGVGGRLYIEQSRELYTWGDQPTLPYELDSSIIEQHPTWQNW
MTECQPGNIWATSDLYKEPHLRVLALAFRSTQIRGLMVIPLHYREQFIGVLSIFRAEFE
TEILWAGRCEQNRRQLLPQLSFEIWREQKKGLAPEWKPEDMTLAQALYDHFSMAIQ
QQQIYK
NpAF142g1 (SEQ ID NO: 73)
FLTNQVSQTQEILLHRIASRIRQSLELQEILSATVAEVRSFLGTDRIKIYQFQADGHGLV
IAESIQEDRLPPLLGLNFPADDIPPYARELFVRARQRCIVDLTTQEIGISPLDCPETGKPL
EQQDIRYRPVDPCHLEYLTAMGVKSSVVVPIVLKNQETGKDSSPNVMESSQLWGLL
VSHHSQARVVTQQELLLIQSVVDQVAIAISQSILLTQVREQARQEAIINKVTEQLHSTP
VAQLQTALEETVAAFNGSGGRLYLLPDDEQTAKLYTFGLQPNQLDIGQEQTPSPLTQ
ELSLVDRAWVSPMERNNEVLLLANLREGSDVLRKGRPIEEHRLWQKYLFASICPPEN
LENPSHKSWSVNWMRAIYALTPPVNELSYDSNLWAIADLYKEPLLRSVAPCFQTTQV
RGLLIVPLQHGSTIVGCLTIFRDEVDIETIWAGCVDTDSRQLMPRQSFAAWRELRTGQ
AQQWSESEIKLAQALGERFATAVKQHRLYEQVQALNAN
Cph1 (SEQ ID NO: 74)
MATTVQLSDQSLRQLETLAIHTAHLIQPHGLVVVLQEPDLTISQISANCTGILGRSPED
LLGRTLGEVFDSFQIDPIQSRLTAGQISSLNPSKLWARVMGDDFVIFDGVFHRNSDGL
LVCELEPAYTSDNLPFLGFYHMANAALNRLRQQANLRDFYDVIVEEVRRMTGFDRV
MLYRFDENNHGDVIAEDKRDDMEPYLGLHYPESDIPQPARRLFIHNPIRVIPDVYGV
AVPLTPAVNPSTNRAVDLTESILRSAYHCHLTYLKNMGVGASLTISLIKDGHLWGLIA
CHHQTPKVIPFELRKACEFFGRVVFSNISAQEDTETFDYRVQLAEHEAVLLDKMTTA
ADFVEGLTNHPDRLLGLTGSQGAAICFGEKLILVGETPDEKAVQYLLQWLENREVQD
VFFTSSLSQIYPDAVNFKSVASGLLAIPIARHNFLLWFRPEVLQTVNWGGDPNHAYE
ATQEDGKIELHPRQSFDLWKEIVRLQSLPWQSVEIQSALALKKAIVNLILRQAEE
Cph2 (SEQ ID NO: 75)
MNPNRSLEDFLRNVINKFHRALTLRETLQVIVEEARIFLGVDRVKIYKFASDGSGEVL
AEAVNRAALPSLLGLHFPVEDIPPQAREELGNQRKMIAVDVAHRRKKSHELSGRISPT
EHSNGHYTTVDSCHIQYLLAMGVLSSLTVPVMQDQQLWGIMAVHHSKPRRFTEQE
WETMALLSKEVSLAITQSQLSRQVHQQQVQEALVQRLETTVAQYGDRPETWQYAL
ETVGQAVEADGAVLYIAPDLTGSVAQHYQWNLRFDWGNWLETSLWQELMRGQPS
AAMEPMAAVQSTWEKPRPFTSVAPLPPTNCVPHGYTLGELEQRSDWIAPPESLSAEN
FQSFLIVPLAADQQWVGSLILLRKEKSLVKHWAGKRGIDRRNILPRLSFEAWEETQK
LVPTWNRSERKLAQVASTQLYMAITQQFVTRLITQQTAYDPLTQLPNWIIFNRQLTL
ALLDALYEGKMVGVLVIAMDRFKRINESFGHKTGDGLLQEVADRLNQKLSPLAAYS
PLLSRWHGDGFTILLTQISDNQEMIPLCERLLSTFQEPFFLQGQPIYLTASMGISTAPYD
GETAESLLKFAEIALTRAKCQGKNTYQFYRPQDSAPMLDRLTLESDLRQALTNQEFV
LYFQPQVALDTGKLLGVEALVRWQHPRLGQVAPDVFIPLAEELGLINHLGQWVLET
ACATHQHFFRETGRRLRMAVNISARQFQDEKWLNSVLECLKRTGMPPEDLELEITES
LMMEDIKGTVVLLHRLREEGVQVAIDDFGTGYSSLSILKQLPIHRLKIDKSFVNDLLN
EGADTAIIQYVIDLANGLNLETVAEGIESEAQLQRLQKMGCHLGQGYFLTRPLPAEA
MMTYLYYPQILDFGPTPPLPKVALPETETEAGQGNVGDRPLPNSLNRENPWTEKLHD
YVLLKERLQQRNVKEKLVLKIANKIRASLNINDILYSTVTEVRQFLNTDRVVLFKFNS
QWSGQVVTESHNDFCRSIINDEIDDPCFKGHYLRLYREGRVRAVSDIEKADLADCHK
ELLRHYQVKANLVVPVVFNENLWGLLIAHECKTPRYWQEEDLQLLMELATQVAIAI
HQGELYEQLETANIRLQQISSLDALTQVGNRYLFDSTLEREWQRLQRIREPLALLLCD
VDFFKGFNDNYGHPAGDRCLKKIADAMAKVAKRPTDLVARYGGEEFAIILSETSLEG
AINVTEALQVEVANLAIPHTVSGTGHVTLSIGIAVYTPERHINPNALVKAADLALYEA
KAKGRNQWLAYEGSQLPHVDGEV
CparGPS1 (SEQ ID NO: 76)
MEWGDLERCEGARIQSWGCVLVVRPSDQFIVWCSERAFEWLGFELDSLSLEPNGIDI
ALIVPEARTVLRNDDFNSWRAVAFTTTKGKQFHCSIHTTEEYYLLEFEEYCAESLAK
MKDSINEASERILCIDNEEELLTQMIKETSELCSFGDRVLIYHFLPATGGAHGRVMAE
NLRCGKEAGWQTFKGLNFPSSDVPFSSRKLFQLTRHRYLAAVSGPQIPLVKIAPGVVS
PPDLSRSFLRGYTNCCQQYYVNMGVKAILVYAIELPGQGLWGFLSFHSTTENYVPIV
KRNALLPLVRTFAGTLKSIKARKRMADESRAKELQLQIFGRLRGQTNFLMGMLDGQ
PSLKDALPCGSCVVFHDDEVLCGTGPLPPVDFILRTMKMIRSSTNPIESTWMTGSLDP
GRLWLTDKLSDLIPEARLISGISAGALLVQVSWRMQVYVMWIRPAVEREVSWAGAP
PGSAYSIDKEGDLNPRASFQKWRELVMDRCEPWDESCLNAAQAVGNTMAEALRIN
KMISSSTKRLNMLAEQMHQIISNVPVAVFR
GwitGPS1 (SEQ ID NO: 77)
MEVDEHTVDTDESHTTPLEFANCIQPHGILFAVRRKDPNLTILQVSKNVWELIGVKVE
DVLGAPFLNLVKPKYRDALFSVLNGSDDVSMSGQRPPFPLRMISNNSKWSCIFYPTD
SGIVVELEPVSHASDDPTQLSFKSSAAGLMKVNAAIERMKSATDVHQLFEISAKELRT
MLGFDRVLLYCLSASGHGRVVAESISDQVKDRVTLYGAQFPARDIPQNAHCMFTTK
MQRLIVDIDQDPSPLVPERNPDTHRILDLSRSVLRGTMSCHIRYVRNMLVKANFVNGI
PKGDGKLWGLLIAHHFSAKFVPYESRASAAQLILALSVLIDNKGEEEYRHFEKQATA
VQIQMLKLAPKEPAEDIMARMTPLLRDVMKCDGLAFVTPQFSVNVGNCPPKASMFA
IAEYLTANDEAISGLWETNCTDNIPTLKGLDLAGCCGILALTISSINKEFLLWFRGAVK
HHVRWSTVSTNEANPLAHIGNREAFVVWRQAVTNCSESWAIADLAVARTLRLAISD
VRTTNEMRLKARMLEEMNRELANSNRQISRVAT
EsPHL1 (SEQ ID NO: 78)
MEMSHSCSGDPERLAQIGHIQGCGYLFAVQETTTGYPSGLRIVTVSQNIMEAPWVQA
SSESDILGKDLGDMLREECVQTVRALVTRYTQASQPRNDRISPKVNRTTADIYSCPA
AAHAGLVPGEEDDPTKAVFTFTLTGSNPGVYLVEVERKHGPHSARAQHTPGLLQMT
DLMGCIPVGSNSLVSTAALCDALAENMPAYERVVVYRFAPDGHGEVIHECIKPGCEV
ESSFLGCHFPASDIPPIARKLLMLKGVSFIADTSGQRVAIRSIPERSSTPLDLSRSALRAP
SPCHEQFLRNMGVRASMAVAITVGGELWGNINFHAYTRAVHPSCEERILAETTVTLT
GALIMHYERQEIATTALALSRMLGRLKDYPRVNDFLSSEHQALRDILEVDTIVLYEHS
RSATVYGKKDISLTLSECIELLNGDGPGETLCFRSLEAKGVAFFSVNTFLIVFLRENIA
DQIKWAGNPEAIMHDGEVVRPRASFQQFMGTPAVPFKLWSPATTDLLNMVRRGFSS
KIYADA
DtenPHY1 (SEQ ID NO: 79)
MAAKISRSTPAINGNLVAQKQSLRDAVLEEEWREGARAAGGNEGAMAKGYDYAGR
IRELRAAEERAREGGDQAVATHSQIEKYTASIFRPGGVQPHGCCVVFDPEMNRIVAL
SRNAPEKLGVSIAPDSMLGRSPSELFGQRTLGILAHAAKSDAVAMDLLCPFPVQSKD
ERDSGMQASLHSTDEGLVMDIEPVDSGVQGLMACHRLIIASAERLRMQSTPSELFQT
MAEEVHALTKYNRVMIYKLHPDAHGEVIAESVDAGVSSFLGLHFPGTDIPQVNRTLF
ECLPKPRLLVDACAPSSEMVQEEHLKQGVLLGASQLRGTVGCHAEYLKNMKVTGTL
VTPVTVVDQEASTQRRLWGLIVCHHEQERFVGYEYRAALSLLIQIFTVRLEKLLQVE
AELNKQSVSSAQNKLMDILLSDPSLRAHELLFGPTAVPELLAIVPGDALCIVSPNTGR
VATGGEYPPSDDLALRLAQWLWSWYKSKRASREKDAMRVFSIPSLAAEGFPEAKDV
AETVAGVLVVVLQLQSGSTTPLATTSDRSEDSSSVLIVWTRREFKEIITWAGGTDKTN
YVQHSAGGSLHPRGSFGHTEEYALTAHPWLKQELDTVQQLGILLQETTESNEASSLR
MDILSSLNEQRFKGMQEVTRLAA
NpyrPHY1 (SEQ ID NO: 80)
MEDGATRYGLCDDDAKDVELSKLYEQQATIALASSKAKLDEEIRDFEAYGRLINRPG
LIQAHGCTILLQKKTLKILASSMNSLDHLGVFLDADSLQDIQVCMQAADPTLTNPRH
VKTVSGKTLHAILHHVKDGTATGVTACFRMIHEAMERIGVIKDLSTLYDGIVKEIQN
LSGYDRVMLYQFHEDNHGEVVAECRKDVNIPSYLNLHFPATDIPQVSRRLFMTMKQ
RMVGDIKASQVGFVQAPELAEENLLLSQSQLRGVTGCHAQYLENIGVRATLVNAVV
VGPTHSSAASAFGTKDNSRKLWGLIVCHNLHDKRIVEFHVRSALLFLVDVFALQLQR
LMDIMETAQNELMWSAQNELYNNVVTTDDFNIGAVADNPECLMKFVGASGAAVLS
GGSLHLIGRTPRAVQLNELADWIRAQPSYDDQRVVCTRSLVADGAPGAASYVQDAA
GLLAVGVPQKATSSVPDSMIIWFRSEVVESVTWAGTNEQHVPKRESDYLQPRESFKA
YLKTMANTSARWEPRILVSVRSFQEVLSLTQTCDNKMKTLAHMTSRVSVQRSITVSQ
LTAVSR
Pco1PHY1 (SEQ ID NO: 81)
MERAGACRARAELAPPAAGEGRAPGLYGARRGAHGRFVFCGRRGLIAVFPADGAD
AAERARLVEKVTERIYRPGVVQGHGAVLILERAHALAFGAHTLAAAPKGGKGQSGG
RAGPTARPPAPRVIAASETCAPMLGVGASELLGAPASRALTSRSLRKICAAAASGAPE
ARTPIAIAPREADARRRSKEHGGALAAAVHVTEEGVVIDLESTHTSPLAPLPGCEANA
DGSHTGAAVGATFGGCGSDSDSDSDSSADDVRDLPPGVSQCHRAAAGAAMRLRAL
RGEATLCEALACEVREISGFDRCMVYRLHADMHGEVCAEARRAESVPSFLHLHWPA
TDLPQVNRRLLTKHPAPRLLADVNDTPSPVLQCAEGDLASAFGCRQALLAGSQIRGT
IGCHKEYLNNMGVRGTLVVPLIIAAAAADSADDAHEDIGGSSSAGGGKPALRMWG
MVVCHNISPRFVPLHTRVCLSMIAQIFTQRLEHELEIARLSTADRNARANSRLVELIIA
DNPRQKATEVLSGSLHTLREAVPFDADGVFVLDSSCTVIACEGAAESGRIEAARVAR
WIKSSGKLHEDAQHPGVFVCDSLEVAGYAALAAAQAVAGGIAGVVAIELPAGVTGT
DGHNGASALEEGACSDPEIAESGGSAVGSPKGRRAIGGMIIWTRAELLQDIKWAGAR
GDSRDVVVIDHDDGAGDDDVKGAPRLGPRSSFAVVLEQLRGTCELWPRIAARGAGA
LVAVLRDAVEAATGSLLRAGIASVLNRSRLSDMGELEKVAA
TastPHY1 (SEQ ID NO: 82)
MGDAAGRARVRAQDRQFLADVELEARFALTDGGCPASAQQPAASAGFNYQDSVEV
TKAANSSAARGIPVSTDQLHAYDRRTLRPGQVQSFGALLLVGHGESQGKCAFPRGL
VLAASANSKEVLGVEASQLLDTFLFNPEVSPFDAECLGALDELLKSRDLTAQAPLLA
TLRLVGGSGAQRQAFLIVHSTEEGYCIDVEPLNEDVSRLLRGSILTHSLAKKGVERLQ
EMSSCSVEEQCQALCEEARALTGYDRIMVYKFHPDCHGEVVAESVSEHVKEPMLGL
HFPATDIPQANRGIFMAMRSRMIADTSASNVKILQSPRLLDNIILAKSQLRSVSGCHAT
YLQNMGSTATLTLSIVTAPTADGFQLKPNYFKMGLPDLSGAKENEGRPPPRSPLWGL
VCCHHSQGPYRVNYDHRSAAEFLVKVFSLQLGRRLDALEAAAQDRVNIAQQSICKV
LKSIEESPHHLEGRTEALADGLMRGHSGKVMMQVAEATGAAMLVGGKWRTTGLCP
QPDQLDTLAAWLCSGQEQAPPRLPHGGRWGTISLHKEGFPNAKAIRGCAAGLLALD
LSRSAASSTGTQCLAIWFRGELMKEQNWAGNKNDPQGRAMGHEMTPRSSFIAMSEI
LDLECRQWADCEVDGTHTIQLLLQDSIRFAEEGMTTGRILIAINQERLRNMDELTKV
AS

1. A food composition comprising:
a food; and
one or more recombinant cyanobacteriochromes (CBCRs).

2. A food composition comprising:
a food; and
one or more isolated or purified CBCRs.

3. The food composition of claim 1, wherein the one or more CBCRs are selected from the group consisting of NpF2164g5, NpR6012g4, NpF2164g3, NpAF142g1, NpAF142g2, NpAF142g3, NpF0020, NpF1000, NpF1183, NpF1883g2, NpF1883g3, NpF1883g4, NpF2164g2, NpF2164g4, NpF2164g6, NpF2164g7, NpF2854g1, NpF2854g2, NpF2854g3, NpF4973, NpF6001, NpF6362, NpR1060, NpR1597g1, NpR1597g2, NpR1597g4, NpR1759, NpR2903, NpR3691, NpR3784, NpR3797, NpR4776g1, NpR4776g2, NpR4776g3, NpR5113g1, NpR5113g2, NpR5113g3, NpR5313g1, NpR5313g2, NpR6012g2, NpR6012g3, Synechocystis Cph1, Synechocystis Cph2, CparGPS1, GwitGPS1, EsPHL1, DtenPHY1, NpyrPHY1, PcolPHY1, TastPHY1, Ava 3771, Anacy_2551g3, Anacy_3174g6, Anacy_4718g3, Apl_4973, WP_016873240, WP_016878855, AFZ15460g3, Cyan7822_4053g2, Cyan8802_2776g1, UYIDRAFT_04680, WP_016871037, M595_0799, Mic7113_2205, Mic7113_2408, LYNGBM3L_56870g6, Nos7524_4790, Fdi_DRAFT46470, Pleur7313DRAFT_05530, WP_033374293, Sta7437_1656, Syn7502_01757, Oscil6304_1286, Oscil6304_4336g2, Oscil6304_4065g2, Oscil6304_4080, Oscil6304_4203, Oscil6304_2705, Oscil6304_3021, Oscil6304_4174, RcaE, Tlr0924, and combinations thereof.

4. The food composition of claim 1, wherein the one or more CBCRs are selected from the group consisting of NpF2164g5, NpR6012g4, NpF2164g3, NpR4776g3, Tlr0924, NpR5113g2, NpF1883g3, NpF2164g6, Anacy_2551g3, Anacy_3174g6, and combinations thereof.

5. The food composition of claim 1, wherein the one or more CBCRs comprise one or more truncated CBCR domains.

6. The food composition of claim 1, wherein each of the one or more CBCRs independently has a length of less than about 230 amino acids.

7. The food composition of claim 1, wherein at least one of the one or more CBCRs exhibits a color change in response to light comprising a photoconversion wavelength.

8. The food composition of claim 1, wherein at least one of the one or more CBCRs exhibits fluorescence in response to light comprising a fluorescence excitation wavelength.

9. The food composition of claim 1, wherein at least one of the one or more CBCRs exhibits a color change in response to a pH having a protochromic pH value.

10. The food composition of claim 1, wherein the composition comprises:
a first CBCR having a light absorption at a first wavelength, and
a second CBCR having a light absorption at a second wavelength, wherein the first wavelength is different from the second wavelength.

11. The food composition of claim 10, further comprising a protein, wherein the first and second CBCRs are each domains of the protein.

12. The food composition of claim 1, wherein the food is a beverage.

13. The food composition of claim 1, wherein the food is not a beverage.

14. The food composition of claim 13, wherein the food is an ice pop, a frosting, a glaze, a shell, or a coating.

15. A food coloring composition comprising one or more recombinant CBCRs.

16. A food coloring composition comprising one or more isolated or purified CBCRs.

17. The food coloring composition of claim 15, wherein the one or more CBCRs are selected from the group consisting of NpF2164g5, NpR6012g4, NpF2164g3, NpAF142g1, NpAF142g2, NpAF142g3, NpF0020, NpF1000, NpF1183, NpF1883g2, NpF1883g3, NpF1883g4, NpF2164g2, NpF2164g4, NpF2164g6, NpF2164g7, NpF2854g1, NpF2854g2, NpF2854g3, NpF4973, NpF6001, NpF6362, NpR1060, NpR1597g1, NpR1597g2, NpR1597g4, NpR1759, NpR2903, NpR3691, NpR3784, NpR3797, NpR4776g1, NpR4776g2, NpR4776g3, NpR5113g1, NpR5113g2, NpR5113g3, NpR5313g1, NpR5313g2, NpR6012g2, NpR6012g3, Synechocystis Cph1, Synechocystis Cph2, CparGPS1, GwitGPS1, EsPHL1, DtenPHY1, NpyrPHY1, PcolPHY1, TastPHY1, Ava_3771, Anacy_2551g3, Anacy_3174g6, Anacy_4718g3, Apl_4973, WP_016873240, WP_016878855, AFZ15460g3, Cyan7822_4053g2, Cyan8802_2776g1, UYIDRAFT_04680, WP_016871037, M595_0799, Mic7113_2205, Mic7113_2408, LYNGBM3L_56870g6, Nos7524_4790, Fdi_DRAFT46470, Pleur7313DRAFT_05530, WP_033374293, Sta7437_1656, Syn7502_01757, Oscil6304_1286, Oscil6304_4336g2, Oscil6304_4065g2, Oscil6304_4080, Oscil6304_4203, Oscil6304_2705, Oscil6304_3021, Oscil6304_4174, RcaE, Tlr0924, and combinations thereof.

18. The food coloring composition of claim 15, wherein the one or more cyanobacteriochromes are selected from the group consisting of NpF2164g5, NpR6012g4, NpF2164g3, NpR4776g3, Tlr0924, NpR5113g2, NpF 1883g3, NpF2164g6, Anacy_2551g3, Anacy_3174g6, and combinations thereof.

19. The food coloring composition of claim 15, wherein the one or more CBCRs comprise one or more truncated CBCR domains.

20. The food coloring composition of claim 15, wherein each of the one or more CBCRs independently has a length of less than about 230 amino acids.

21. The food coloring composition of claim 15, wherein at least one of the one or more CBCRs exhibits a color change in response to light comprising a photoconversion wavelength.

22. The food coloring composition of claim 15, wherein at least one of the one or more CBCRs exhibits fluorescence in response to light comprising a fluorescence excitation wavelength.

23. The food coloring composition of claim 15, wherein at least one of the one or more CBCRs exhibits a color change in response to a pH having a protochromic pH value.

24. The food coloring composition of claim 15, wherein the composition comprises:
a first CBCR having a light absorption at a first wavelength, and
a second CBCR having a light absorption at a second wavelength, wherein the first wavelength is different from the second wavelength.

25. The food coloring composition of claim 24, further comprising a protein, wherein the first and second CBCRs are each domains of the protein.

26. A method of coloring a food, the method comprising:
adding a food coloring composition of claim 15 to a food.

27. The method of claim 26, wherein the food is a beverage.

28. The method of claim 26, wherein the food is not a beverage.

29. The method of claim 28, wherein the food is an ice pop, a frosting, a glaze, a shell, or a coating.

30. The method of claim 26, further comprising:
subsequently illuminating the food with a light comprising a photoconversion wavelength, wherein at least one of the one or more CBCRs exhibits a color change in response to light having the photoconversion wavelength.

31. The method of claim 26, further comprising:
subsequently illuminating the food with a light having a photoconversion intensity, wherein at least one of the one or more CBCRs exhibits a color change in response to light having the photoconversion intensity.

32. The method of claim 26, further comprising:
subsequently adjusting the pH of the food to a protochromic pH value, wherein at least one of the one or more CBCRs exhibits a color change in response to a pH having the protochromic pH value.


SEQUENCE LISTING (82 sequences)

SEQ ID NO: 1 - protein, 176 aa, Unknown
  Unknown Cyanobacteriochrome NpR6012g4
EKAVTKISNRIRQSSDVEEIFKTTTQEVRQLLRCDRVAVYRFNPNWTGEFVAESVAHTWVKLVGPDIKTVWEDTHLQETQGGRYAQGENFVVNDIYQVGHSPCHIEILEQFEVKAYVIVPVFAGEQLWGLLAAYQNSGTRDWDESEVTLLARIGNQLGLALQQTEYLQQVQGQSAK

SEQ ID NO: 2 - protein, 174 aa, Unknown
  Unknown Cyanobacteriochrome NpR4776g3
QQRVLFEVVAKVRKSLDLDAIFQTTTQEICKSLQADRVAVFQFQADWSGEYIAEFVGDGWVKLVGSNTKTVWQDSYLQETQGGRYRHNETFAVDDIYQVGHSQCHVAVLEQIQARAYAIAPIFIGQQLWGLLAAYQNSAPRHWEASEIKFITQIANQLGVALQQAQLHNQTKEQ

SEQ ID NO: 3 - protein, 170 aa, Unknown
  Unknown Cyanobacteriochrome Tlr0924
EREMIVSTIIQDIRQSIRLEEILQRAVNSIQQLLLSDRVLIYRFLGDGSGIVAVEATTLPQYSILGQVIHDPCFTKETARRFLEGRTLSISDVNQAQLQDCYRELLTRLQVQANLVVPLLQGQHLWGLLIAHHCRSPRLWQREELFLLQRIAEPLTVALQQAEMYEALEQ

SEQ ID NO: 4 - protein, 192 aa, Unknown
  Unknown Cyanobacteriochrome NpR5113g1
PNSNFAENQLLLSSAQLQEQLDQQKALASVIVRIRESLDLETIFQTTATEVRLLLNADRVAVFQFDLEKDWEGEFVSENVAPGWTSALKVKVYDHCFGEQFASSYQQGRVQAVADIYNAGLSDCHAAILSKFQVKANLVVPLSRQKELWGLLCIHQCSEARNWKESEIEFIRQIADHLTVAIQQAKHLHEVQ

SEQ ID NO: 5 - protein, 168 aa, Unknown
  Unknown Cyanobacteriochrome NpR5113g2
RDQVIAQIVDKIRSPLDIETIFKTTTQEIRKLLQADRVAIFSFNADWSGNFVAESFAEGWTPLVGIQPTIFDTYLQQTQGGRYVDNETFTVNDIYQAGLTDCHVTLLEQFQAKAFATAPILQGDKLWGVIAAYQNVSPRQWQSYEVESLTKIGTQIGVALRHYKLLAH

SEQ ID NO: 6 - protein, 167 aa, Unknown
  Unknown Cyanobacteriochrome NpF1883g3
MQSQLLADTIGSIRQSLNEEDVLKTTVEEVRKVLSTDRVMIYSFNANWSGTVIAESVVLTYPKVLRAEIEDPCFGQGYVKEYQSGRVLAINNIYEVGLADCHINLLESFGVKANLVAPILKDEQLFGLLIAHQCSRPRDWKQSEIDLFAQIAMQVGFALDHARLLQR

SEQ ID NO: 7 - protein, 202 aa, Unknown
  Unknown Cyanobacteriochrome NpF2164g3
ERERTVFGIIEKIGQATDLRTLFRTATQELRRFLKCDRVVVYQFHPDWSGEVIAESVGAGWSSLVEMQEQDGILKTGLISSERCNIKDYGSPVKADADTYLKETQGGMYAKGTRFRKVNDISAAGFSSCYLESLEKFQAKAYIIVPVFRAGNLWGLLAAYQNSEVRQWTEEEAGALLQIAEPLSIALQQAEYIEQLRLKNLE

SEQ ID NO: 8 - protein, 170 aa, Unknown
  Unknown Cyanobacteriochrome NpF2164g5
EEQVLTAMVEKIRQAQDMDTIFLNVLPSLRKQLQCDRLAVFRFHPDWSVEFVAESVKDKWLSLADSDIKTIWMDEHLQETQGGRYRNHETFVVNDIYTVGHVQCYLEILEKIQAKAYAIAPIFIGNKLWGFIGAYQNTGPREWKSKDVQLLRKVAVQMGIGLQQVKYIEQ

SEQ ID NO: 9 - protein, 169 aa, Unknown
  Unknown Cyanobacteriochrome NpF2164g6
ERALTRLSEKIRQVQELDTIFRNALPELRSHLECDRLAVYRFNPDWGGEFIAESVSREWVALVGPEIRTIWEDEHLQQTQGGRYRNNETFVINDVYTAGHAQCHLKILEQFQIRAYIITPIIAGNKLWGLLGAYQNSGSRQWQENEVNLVAKIGTQFGVAVQQSQYLQQ

SEQ ID NO: 10 - protein, 189 aa, Unknown
  Unknown Cyanobacteriochrome Cyan7822_4053g2
EQIRQKLRQQQAIAAIVQQVRQSLNIEEILNTITQDVRALFDCDRVIIFRLYSDGGSRIIEESVSTEFLPLKYCHWDDETWSQDILNLYWQGQPRIVPDVMNDIYTECLHEYSREGQIQSKIVAPILLDLKEKENHRWVASTNSHKLWGILVVHACREKRVWQNSEAQLLQQIANQLAIAIQQASLFEQ

SEQ ID NO: 11 - protein, 176 aa, Unknown
  Unknown Cyanobacteriochrome Sta7437_1656
ERLITTITQNIRQSLDLKKILATTVIEVQQTLNAERVLIFRMNPDGSGQVIEEAVVPKYPVTDQMRWEDEHFPEDCYEYYRQGIPRIVPDVATDEWAKCLVEFMQEVGVKSKVVAPIVQVYEKSSTNAKVWGLLIVHACSHYRQWQESEVDFLQRIGNQLAIAINQANLYQQLQAE

SEQ ID NO: 12 - protein, 192 aa, Unknown
  Unknown Cyanobacteriochrome Anacy_2551g3
EDAISLQLQRQKIIQDITQQIRSTLNVNHILATVTQQVKELMQVERVIIFRLFPNGRSQIVEEVVSSEYAALKNYHWEDEKWSQEILDCYWQGKPRIVPDVINDIWTSCLVEYTTQGNIQSKIVAPILQELGENETGRWVSSEHKQKLWGVLVVHACSTKRVWEEDEAQLLQQIANQLAIAIQQAALFEQLQ

SEQ ID NO: 13 - protein, 193 aa, Unknown
  Unknown Cyanobacteriochrome Anacy_4718g3
ELAISHQLQQQRTLGKIVQKIRDSLDIKDILATVTQEIKNSLNCDRVIVFRLFADGESQIVEEAVSGELVSLKNRHWDNEVWSQEILDYYWQGQPRIVTDVMEDIWTDCLVEYSIEGQVQSKIVAPILQEAQDGEKNRWVASGENNKLWGVLVVHACSEKRIWKDCEAQLLQQIANQLAIAIQQANLFEKLQQ

SEQ ID NO: 14 - protein, 174 aa, Unknown
  Unknown Cyanobacteriochrome WP_016871037
ERLLITITQHIRQSLDLEQILRTTVVEVQRTLQTDRVLIFRLNQDGSGQIIEEAVVPEYPMTYQMRWVDECFPDDCYEYYRQGNPRILPDVAKDEWGACLVEFMQQIGVKSKVVAPIIQTLEDSSTRVWGLLIVHACSHYRQWQASEAEFLQQISNQLAIAIHQADLYYQLQIE

SEQ ID NO: 15 - protein, 205 aa, Unknown
  Unknown Cyanobacteriochrome NpR4776g1
MTSNPEQNFLYSQEESLLRRITNRIRRSLELEEIITVTTAEVRSLLKTDRVMIYKFHADGNGQVIAESIYNNRLPSLLGLNFPADDIPLSARELFLKLRVRSVVNVDTQEIGQIHLRDLDNGETISEEIRYRSVDSCHIEYLTAMGVKSSVVAPILYQDQLWGLLVSHNSEARLISEYELEAVQMVVEQLSVAIAQSSLLTQVRK

SEQ ID NO: 16 - protein, 172 aa, Unknown
  Unknown Cyanobacteriochrome WP_016878855
QQERLLGTITQHIRQSLNLEEILATTVIEVQQTLQADRALIFQLNQDGSGQIIQEAVIPDYPVTNQMRWLDECFPDECYEYYCQGNARIVPDVAKDDWGACLVEFMQEVGVKSKVVAPIVQSFEGSSNKVWGLLIVHACSHYRQWQASEVEFLQQLCNQLAIAIHQANLYHQ

SEQ ID NO: 17 - protein, 198 aa, Unknown
  Unknown Cyanobacteriochrome WP_033374293
EAMIAQQLRQRTVLGQIVQQIRESLNLQEILAITTQRVREILQGDRVIVFRFCDLGRTCIFEEAVAEDLPSLKYMNWEDEQWSSEILQFYWQGQPRIVPDVMNDPLTPCLLDYSRQGQIQSKIVAPILQEIHNGERGNGEIDPWTDPESGNKLWGLLVIHACHEKRIWQESEAELLQQIANQLAIAIRQSRLFEQLQE

SEQ ID NO: 18 - protein, 197 aa, Unknown
  Unknown Cyanobacteriochrome WP_036801164
RRRQAEIFLWKQSERERVINQITQQIRQSLNLDEVLSTTVKEVRKFLDCDRVLIYRIREDGTGSTITETVLSPYPAILGQTFPEEVFPTEYHQAYIQGKSRTITNIEQDDVEECLADFVKQFGVQAKLVVPIIQETRDISLSEEKQTTAPYLWGLLIAHQCSQTRQWQPLEVQLMQQLATQVAIAIQQSELYTQLQQ

SEQ ID NO: 19 - protein, 185 aa, Unknown
  Unknown Cyanobacteriochrome O6304_4080
QAEEAMRQQVERERLLGAIAQRIRQSLDLTTILSTTVEEVQQVLGCDRVLVYRVWADGTGSAIAEAVKPGWLKVLNRVFPEEVFPPENYQRYIEGRICALVDRDSGQTLPCLVEFMKSIEVRAKLVVPIVEKDTLWGLLIAHQCSGPRQWQGWEIDLLASLATQLAIAIQQSELYERLQVELSDR

SEQ ID NO: 20 - protein, 188 aa, Unknown
  Unknown Cyanobacteriochrome UYIDRAFT_04680
EQSLQLQAEQERLLSTITQHIRQSLDLEQILRTTVVEVQQTLQTDRVLIFRLNQDGSGQIIEEAVVPEYPMTYQMRWVDECFPDDCYEYYRQGNPRILPDVAKDEWAACLVEFMQQIGVKSKVVAPIVQTLEDSSTKVWGLLIVHACSDYRQWEASEAEFLQQISDQLAIAIHQADLYQQLQIELAER

SEQ ID NO: 21 - protein, 182 aa, Unknown
  Unknown Cyanobacteriochrome NpF3797
ERERLVNQIAQHIRESLELDEVLTTTVAEVREFLQADRVLIYRLWEDGTGSAITETVLPEYTKILGETFPEEVFPREYHQAYSLGKTRAIANVEQADVESCLADFVKQFGVKAKLVVPILQENREADKQRDSKTPASPPYLWGLLIAHQCDRTRVWESWEIELMKQLATQVAIAIQQSELYK

SEQ ID NO: 22 - protein, 178 aa, Unknown
  Unknown Cyanobacteriochrome RcaE
QRVELFSEVTLKIRQSLQLKEILHTTVTEVQRILQADRVLIYHVLPDGTGKTISESVLPDYPTLMDLEFPQEVFPQEYQQLYAQGRVRAIADVHDPTAGLAECLVEFVDQFHIKAKLIVPIVQNLNANSQNQLWGLLIAHQCDSVRQWVDFELELMQQLADQISIALSQAQLLGRLEE

SEQ ID NO: 23 - protein, 176 aa, Unknown
  Unknown Cyanobacteriochrome WP016873240
KREQLLRGITQRIRQSLDLEQILATTVNEVLQTLQSDRALIFRLHGNGTGQVIQEAVRPEYPVTEQMLFPDECFPQECYEYYCQGQPRIVSDVFAEDFSSCLVEFMQKIGVKSKIVAPIVQTTENSSTKVWGLLIVHACSQHRQWQQSEADFLQQISNQLAIAIQQSQLYQQTRQQ

SEQ ID NO: 24 - protein, 175 aa, Unknown
  Unknown Cyanobacteriochrome Nos7524_4790
RERLLRNITQHIRQSLDLESILSAAVNEIRQTFQAERALIFRLMPNGSGVVLEESVVPDYPSIKSRLWQDECSPHELYQFYRDGNVRIVTDTQNDLWGECLAEFMAEAQVQSKIVAPIAQKLDNQPPRVWGLISIHACGKKRQWQTDEANLLQQIASQLSVAIQQADLYQQVQTE

SEQ ID NO: 25 - protein, 188 aa, Unknown
  Unknown Cyanobacteriochrome Syn7502_01757
ERQNFITKITQQIRQSLDLEEVLTAAVNEVSSLLNCDRIIIFQLFPNGASQVVKEIVKPPYPSIIGMNWQDEHFSHEGFEYYINSNPRTVNDVFEDNVFADCLKEFMSQAQIKSKIVAPIIQHIQSCSLEPYNRWRRDNSQLWGLLIVHSCGEYRQWDQEEADLLQQVANQLAIAIQQADLYQQVQQE

SEQ ID NO: 26 - protein, 217 aa, Unknown
  Unknown Cyanobacteriochrome NpF1000
KQISQERLLNQVTTQIRKSLDLPVIMATAITQVREFLELDRLVIYKFEASKVKTQEYQSSTDEDNGKGSTSISVNNQSLLEDYQHRGYIVYEARATDAITCVLNYTEKNCFMRTSQCWEKYRQGFTLAVDDVEKTYALEECLLNFLRESQVRAKLAAPIILENKLWGLLIAHQCNEPRQWIDSEKNLLIAIAEQLAIAIYQAELMQTLTQEKQTLEH

SEQ ID NO: 27 - protein, 208 aa, Unknown
  Unknown Cyanobacteriochrome O6304_4203
REMLLSRISAKIRSTFDLPTILKLGVQGLRQALDTDRALAFQFLADGSGVCIAEDVQAPYPSIRGQWFPADCMPLTHRDAYRNGHLGSVPDIQNAALSDCYREMLNQFQVRSFMGVAIGRNQEGSIIPDSLSFKELQNSTGEISEIDSNVLKSPSVSSLWGLLVVHHCHGPRQWTMDEKQLVQAVATQMAIAIEQANLVQQLRTYARE

SEQ ID NO: 28 - protein, 174 aa, Unknown
  Unknown Cyanobacteriochrome O6304_4336g2
ELQQSLKFTRILKQITDQIRSTLDVSTILQTIAQRVRNLLNTDRVVIYQFDDMGNGEVTVEDIRESWQSVRGVRCPGCIPPDVSNLYLKGRIRAINNVATAELSACHREFLDSIQVKANLIVPICMGSDLWGFLIAHECEQPRLWQEQEIDLLQQLADQAAIAIAQAELYQQTQ

SEQ ID NO: 29 - protein, 173 aa, Unknown
  Unknown Cyanobacteriochrome NpR5313g2
TKVIKQITEQIRRTLDLQTTLQTIVSEVRSLLNSDRVVIFQLNSKSVIVEEMNGNWQSVLGVNAPPECFPNEHRDLYSQGRVRAINNVSTDSLSDCHREFLQSLQIQANLTVPINIGIELWGLLIAHECNTPRNWQDVEIDLLQQLGDQAAIAIQQAQLYEQTCKAETEARNK

SEQ ID NO: 30 - protein, 188 aa, Unknown
  Unknown Cyanobacteriochrome O6304_3021
ERERVMSAMRDRIRSSLNLKEILNTTVAEVRQFLNTDRVIIYRFHPDWSGKVVVESCGTRAREILNLTILDPCFGETHAQLYKKGRLKATPDIYTEPISPCHRNLLAQFQVKANLVVPILLNEDFSHNSEEELLENSVGDNNQLWGLLIAHHCQGPRQWQNWETEFLQELATQVAIAIQQSTLFEQLQ

SEQ ID NO: 31 - protein, 178 aa, Unknown
  Unknown Cyanobacteriochrome NpF4973
MQIHPHSEFNHSRDRREQGLQNLLDRLVKSIQRDELVRQTTNQLRESLQVDRVVLYYFYSQWQGQVTFESLSSKEFSILSSTGPDDCFNNEYAALYLAGRVRAIANIELEPIQPCHRDFLRNLQVRANLVVPIVIPRGLWGLLVAHHCQGPHDWSSSDIEMMQTGAQTLATDRNILES

SEQ ID NO: 32 - protein, 179 aa, Unknown
  Unknown Cyanobacteriochrome Apl_ORF1
MNFPNIFGDSTNSNPNPNSDPRFQQFVERLQQVEDRDRLIQETLNGLRKHLKSDRIVLYYFYKQWRGQVTFESLRYIDYSIYGSTGADDCFNDQYARLYLEGRISAISDIDEADIDSCHRDFLKSIRVKANLAVPVVKNQQLWGLLIAHYCQTSHSWVNAEIDTMKQAAKLLSESPFLD

SEQ ID NO: 33 - protein, 164 aa, Unknown
  Unknown Cyanobacteriochrome O6304_2705
MSDWSLRQVADRLAKTLNQDLLIRQTTLELREILNVNRILLYYFYSPWKGQVTFESVSAPQFSIIGQSGPDQCFGDEYAALYLQGRVRAISNIETEPINECHREFLRTLQVKANLVVPVLSPIGLWGLLVAHHCEDTRLWSLDDIKRMKNAAETLGTAATIRDS

SEQ ID NO: 34 - protein, 165 aa, Unknown
  Unknown Cyanobacteriochrome NpF1883g2
AQIFIEITRSIRESLNEEDVLKTTVEEVRKVLSTDRVMVYTFNANWSGTVIAESVIPGYPKVLRSEIQDPCFGQGYVEKYQSGRVATTNNIYQAGLADCHISLLESFGVKANLVAPILKDEQLFGLLIAHQCSRPRDWQQPEIDLFAQIAIQIGFALDHARLLQR

SEQ ID NO: 35 - protein, 175 aa, Unknown
  Unknown Cyanobacteriochrome NpF1883g4
RSQLLVDITRSIHQSLKEEDVLKTTVEEVRKALSTDRVLVYSFYANWFGIIIAESVVPGYPKVLRSKIHDSCFTQGYIEKYQSGRVVAINNIYESGLADCHIKLLESFAVKANLVAPIIKDDQLFGLLIAHQCSGPRDWQQPEIDLFTQIAMQVGFTLDYARLLQAYQATEASSN

SEQ ID NO: 36 - protein, 213 aa, Unknown
  Unknown Cyanobacteriochrome NpR1597g1B
MENSSTTQPIEPNSEQQESHSAVTSSLRQNEQQKALSGVIARIRESLDIETIFKITVTEVRQLLKTDRVGVFRLYPDLAWEGEFIYEDVATEWDSALAAKLCDHCFSEEFASLYQQGRIRAIADIYQVNASDCYIQILERFQVRANITAPLIKGKNLWGLLCIHQCSSSRQWEASEIEFVQLIAEHLGVALQQADYLEQVKLQSAQLAQAKAR

SEQ ID NO: 37 - protein, 183 aa, Unknown
  Unknown Cyanobacteriochrome NpR5113g3
EQQKTLTSVITRIRESLDLDTIFQTTVTEARKMLQADRVAVFRFDPQKDWEGEFISEDVVPECNSVIAEKIYDYCFGENFASLYAQGRVNAIADIYQAKFPGCYIQILEKFQVRANLVAPLLKKGELWGLLCIHQCTTFRHWQPSEIEFTSQIAEQLGVALKQDSYLKQVQMQVVQLAEATER

SEQ ID NO: 38 - protein, 168 aa, Unknown
  Unknown Cyanobacteriochrome NpAF142g3
QQTLASVITKIRESLDLNAIFETTTQELRRVLNCDRVVIFRFYSESNYDGGEVIAEDVAERFLSTLTAKVYDRCLGQEHIEKFCQGYVHAVTDIYNSELNECYVSMLSRFQVRANLVIPMLKQGQLWGLLCIHQCSRPRAWQDSEIEFVRQIAAQLGVALQHVVLLNQ

SEQ ID NO: 39 - protein, 175 aa, Unknown
  Unknown Cyanobacteriochrome NpR2903
LQNLRSQLFANITLKIRQSLQIDEIMQTSVKEVQKLLLADRVLILRLQPNGSFIAVQEAIVPGLPVVLGQQITDPCFRDDYIEQYRQGQINVINDIKEADIQPCHVELLQRFAVKANLVVPIFLKNQLWGLLIAHQCAHPRQWTSWEIELLRQLADQIGIASTQSLILEQETRQR

SEQ ID NO: 40 - protein, 176 aa, Unknown
  Unknown Cyanobacteriochrome NpF6362
KRQNLRSQLFAEITLKIRESLQIDEILQTTVTEVQKLLQADRVLIFRLEADGSGTVVQEAVLPGWPVILRKNLFDPCFKEEYIERYRQGRVSAMEDIETAHIQPCHREFLQQFAVRANLIVPILVRDRIWGLLLAHHCAAPRKWNNFETELLKQLANQIGIALSQAELLEKETQQS

SEQ ID NO: 41 - protein, 172 aa, Unknown
  Unknown Cyanobacteriochrome NpR1060
ERIVAAVTQRIRQSLELDEILQTTAAEVRQLFEVDRVIIYQFESDWSGLVAVESLAEECMSILGFHVMDTCFKSTRAIYFQQGNTRAIEDVETAGLSPCHLDLLRSLQIRANLVVPILQKERLWGLLIAHQCRNARQWQQSEINLFNQLAGQAAIAIQQSELYHQLQQANQE

SEQ ID NO: 42 - protein, 182 aa, Unknown
  Unknown Cyanobacteriochrome NpF6001
EREQLMRTVAQRIRQSLNLQDILNATVQEVRDLLGVDRVVVYQFDSEMIGTIMAESVEPGWRVSLGVEIYDTCFQTGKGAEYYQVNKRAIANIYEAGLTDCHIRLLEQFEVKANLVVPILLEVSSQTPGSHLWGLLIAHQCSGFREWEKNQLDLLDQVTVQLAIAIQQSSILEQAQTELVQR

SEQ ID NO: 43 - protein, 170 aa, Unknown
  Unknown Cyanobacteriochrome Mic7113_2205
QREQLLGEMQGRIRQSLDLEEILSTTVSEVRQFLQTDRVLIYRFFDDWSGVVAVESVSTDELSILNTTISDPCFGEAYVERYQQGRIMVIEDIYTAGMTPCHVDFLASIQVRANLVVPILQKERLWGLLVAHHCYEPRQWQQWEVDFLEALSTQIAIAIQQGQLHQRLYS

SEQ ID NO: 44 - protein, 170 aa, Unknown
  Unknown Cyanobacteriochrome Cyan8802_2776g1
ELQTQDFTLSVTDGENTLKPLENWFSRALAKARQLLGADRLLIYRFAPDGRGTIDYESLGSGVSSTLNQQIGDACIPAELLEAYRVDRVAMINNLSEVTLDPDHLELLTRLGVQANLVIPILNQGNLFGLLIAHQCFSPRQWEERDINFLKQLSTQFQVILNRLSLSHQQ

SEQ ID NO: 45 - protein, 202 aa, Unknown
  Unknown Cyanobacteriochrome NpR3784
MGNNDEFNSMCHLTECQQAEEALQQQIQWQRLVMAIAQRIRQSLNLDKVLNTTVTEVRQFLQVDRVFMYQFEPDYSGVVVVESVDDRWIAILNTQVQDTYFMETRGEEYSHGRIQAIADIYTAGLTECHRDLLTQFQVRANLAVPILQGKKLWGLLVANQCAAPRQWQTWEIDFLKQLAVQVGIAIQQSQLYEQVHTELAER

SEQ ID NO: 46 - protein, 196 aa, Unknown
  Unknown Cyanobacteriochrome Ava_3771
NHDEYNQESLKNVNKQAQEALQQQIERQRLVMAIAVRIRESLRLNEVLNTTVAEVRQFLQADRVFIYRFAPNYSGCVVVESVGDNWRAVLNAQVEDTYFMETHGEKYRQGGLQAIADIYAEDISECHRDLLIQFQVKANLVVPILQGEKLWGLLVTNQCGTSRHWQSWEIDLLQQLATQVGIAIQQSELYEQVRAE

SEQ ID NO: 47 - protein, 195 aa, Unknown
  Unknown Cyanobacteriochrome Mic7113_2408
CKASLEESCQHSEEALRQQFLWEQLLGAIAQRIHQSLNLDEILNTTVDEVRQVLACDRVIIFRLHPDGSGVIVVESVDSGWRPISGISINDHHFAQAYIKLYKQGRVQAVEDIYTADLTPCHRDLLAQLQVRANLVVPIVHEEQLWGLLVAQQCSKPRMWKSLEIDLLSSLATQAAIAIQQSELYQQAQTEIAQR

SEQ ID NO: 48 - protein, 171 aa, Unknown
  Unknown Cyanobacteriochrome WP_023064634
QRQKILFEIVNRIRQSLDLQTIFDTTTQEVRRFIKSDRVVVYRFNSDWSGYFVAESFSSEWKPLIDLIPNIADTCLQETEGGRFRRGEILAIEDIYDAGYSDCQIKLLEQFSARAYIVVPILQGENLWGLLAAYQNTATRRWTDEELQLLSQISIQLGVALQQAELLAETQ

SEQ ID NO: 49 - protein, 173 aa, Unknown
  Unknown Cyanobacteriochrome Anacy_3174g6
EQEKAISKIVNRIRQSLDVEEIFKTTTQEVRLLLKCDRAAVYQFTSDWGGEFVAESVVSGWVKLVGPGIKTILNDPCLQNNEGGRYKKGDTLIVNDIYTATLDPCYIEIIEKYEARAYIIAPILFGEKLWGLLAAYQNSGTREWEESEINLLSRISDQLGLALQQTEYLQQVQ

SEQ ID NO: 50 - protein, 179 aa, Unknown
  Unknown Cyanobacteriochrome NpF2854g3
RQRALTEVVGKIRSSLNIDLILKTTCQEVCKLLRVERVGVYRFNEDWSGEFVSNFGMVEAQWDSINPFGKNLVWEDTHLQETKGGRYRNNENFAVEDIYQAGHSRCHLDILEQFKIRAYALTPIFVGRNLWGLLAAYQHSAPRQWDSVEVEFLGQVASQLGVALQSSQMVTQIQTRADE

SEQ ID NO: 51 - protein, 167 aa, Unknown
  Unknown Cyanobacteriochrome NpR1597g4
REKALAATVEKIRQSLDIGTIFATSTEEVRRLLEVDRVTIYRFRADWSGEFVAESLAQGWTPVREIVPVVADDYLQETQGRNFANGKSIVIKDIYSANYSICHIALLELMQARAYMIVPIFQGEKLWGLLAAYQNIKPRDWQEDEVDLVMQIGTQLGVGLQQAELLE

SEQ ID NO: 52 - protein, 201 aa, Unknown
  Unknown Cyanobacteriochrome NpF2854g1B
ITNSPKTSNLLDLQECHKQLQLTVQYQKILARILAKIRASVNIESICSTSCQDICRQLKIERVAIYRFNTDWSGSFINRFGFAEHPWDALTAFGQDLVWQDSHLQETQGGRYRKNEPFAVADIYDAGHARCHIEVLEQFQIRAYAIAPIFIGAKLWGMLAAYQHSAPRQWWPHEVEFLAQAASHLGVAMQQAETLEQTKQR

SEQ ID NO: 53 - protein, 175 aa, Unknown
  Unknown Cyanobacteriochrome NpF2854g2
RQRALTEVVGNIRSSVNPEIILDTACQELCKLLKLERAAIYHFNEDWSGEFVSQFGMLEPQWHRIHPFGKNLVWQDTHLQETKGGRYRNNESFVVNDIYQAGHSRCHIDILEQFKIRAYTLAPIFIGPKLWGLIAAYQHSGPRQWANYEVEFLGQVGAQLGVAIQQAENLAQSKQ

SEQ ID NO: 54 - protein, 175 aa, Unknown
  Unknown Cyanobacteriochrome NpR6012g2
KKSVAKVIDKILRASNVDKIFKTTTQEVRQLLKCDRVAVYRFKPDWSGEFVAESVGNGWVKMVSPDFYMVWEDSHLQDTQGGRYAKGESFVAKDIYKMGHAQCHIDILEQYEMKAYIIAPIFAGEKLWGLLAAYQNSGPRDWQPWEESFVTQIGLQFGVAISQGEYLEQMHKKSE

SEQ ID NO: 55 - protein, 176 aa, Unknown
  Unknown Cyanobacteriochrome NpR6012g3
EKAFTKIVGRIRQSLDVDSVFKTTTQELRQSLRCDRVAVYRFNPDWGGEFVAESVGTGWTKLVGPDIKTVLDDTYLQETKGGRYVRGENFVVNDIYQVGLAPCHIEILEQFEAKAYIIVPIFFGDKLWGLLAAYQNSRPREWETWEVTFLVQTSLQFSLAKSQIDYLELVRLKSEK

SEQ ID NO: 56 - protein, 180 aa, Unknown
  Unknown Cyanobacteriochrome NpAF142g2
ERQQALARIIANMRQSLDVTTIFRTTTQEVCQLLECDRLSVYRFNADWGGEFVGDYETANPRWGRSIKLGVGMVWDDTYLQETQGGRYRNNETFVVDDIHSQGFTQCHIEILEQFHVQAFMIAPIFVGQELWGLLGAYQHSSTRHWQASEIEFFTQIATQLGVALQQAEYLEQVRAQTRK

SEQ ID NO: 57 - protein, 184 aa, Unknown
  Unknown Cyanobacteriochrome NpR4776g2
RTVELQRTAEQEQAVFKVIAEIRESLDTDTIFQTTTKEVCQLIKADRVSVYRFDSNWGGEFVGDFEAASPYWSNESEIGINTVWNDTYLQDTEGGRYRNNETFAVDDIYKMGFAKCHIDNLEQFQIHAFVLAPIFVGQKLWGLLATYQHTGPRQWKASEVNFLSQIAAQMGVALQQAELLTQTR

SEQ ID NO: 58 - protein, 168 aa, Unknown
  Unknown Cyanobacteriochrome NpF2164g4
ENAIARMADRMRNSQQLETIYRTTLAELRQLFKCDRLAIYRFNSDWSGEFIAESVGSDWVPLVGPDIKTVWEDEHLQQTQGGRYRNNETLAVNDIYTVGYAQCHIDLLEQFQVKAYTIAPIFAGNKLWGLLGAYQNSGPRQWNSEEVRLLTKLGIQLGLSVQQVEYIE

SEQ ID NO: 59 - protein, 186 aa, Unknown
  Unknown Cyanobacteriochrome NpF2164g7B
MEREVANARFSYRLPSRLTEMAQSHGNVLEFIQFATHELRQLLKVDRVGVYRFEPDWSGEFVVESVTGDWPKLVGTSLAKVRDTYLQYNQGGRYVRKESLRVDNIYSVGHDECHIQLLEMWGTKAYMISPIFQENRLWGLMGVYQNSASRQWEQSEEDVLNQASVQIGIALNLADYLTQVRTQEQQ

SEQ ID NO: 60 - protein, 197 aa, Unknown
  Unknown Cyanobacteriochrome Moorea_g6
ERVVASLSNHLSQSVDVYSVFQSITKELRQVLGADRVVIYRFNSDWSGEFVAESVAPGWLSLITEQERDDRLKVDTTASERCTVTVLSVNSLSNVDTCLQETQGGDFARGDRFKQVDDIYAIGFSPCYINTLEQYQARAYLIMPIFQGEKLWGLLAAYQNTGPRQWQDTDVTLMLQISNSLGLALQRSHYLDQLRAQ

SEQ ID NO: 61 - protein, 204 aa, Unknown
  Unknown Cyanobacteriochrome NpR3691
ERAIAQVIQRMRQTLDLETIFAATTQELRQVLNCDRVVVYRFNPQWNGEFVSESVGNGWISLIEEHNNDPNLTEGVLQQGRCLAKILDSGDNQAEDADMQAIQDGIYAQGATFRCVPDIYNAEFHPSYISLLERFQAKAYIIVPILYGSQIWGLLASYQNSDSRQWKPGEINIVVQIGNQLGVALQQAQLLAQTQRQSQALQEA

SEQ ID NO: 62 - protein, 196 aa, Unknown
  Unknown Cyanobacteriochrome fdiDRAFT46470
ERERAIAQVIQRMRQTLDLETIFAATTQELRQVLDCDRVVVYRFHPDWTGEFVSESVGFGWSSLLEAQKNDPYLTEGALAEGRCIWTMLDSLQQVRDSDLNTIPEIEYTQVANFRYVADIDKVKFQPNYINFLKRLQAKAYITVPILCGNEVWGLLASYQNSVPRQWKTEEINIVVQIGNQLGVALQQAQLLAQTQ

SEQ ID NO: 63 - protein, 193 aa, Unknown
  Unknown Cyanobacteriochrome NpR1597g2
KAAQWQKTIAITIEKIRQSLDLESIFRTSTVELRQLLNADRVAIYRFNPDWSGEFVFESVAEGWISLIDEQLQRPELSENVSECSAKDLAKPPVADTYLQDTEGGSFSSCEVYRICNDIYNSGFSDCYIKILEIYQARAYVIIAIYHGQKLWGLLAVYENARTRDWQKDEVYLLTQVGTQLGVALQQAEFIQQ

SEQ ID NO: 64 - protein, 206 aa, Unknown
  Unknown Cyanobacteriochrome NpF2164g2
EKDQILVGVANKILQSKDADTIFRVATQEVRQSLKCDRVAIYRFQPDWSGEFVAESVGSEWVSLLEEQRNNPAIVNNVNYCSVRNLADEGGLAGTTGRSHSADSYIQHTQGKNFHRGQIYRVTNDIYSAGFSDCYIKILEAYQAKAYIIVAIFQGEKLWGLLAAYQNSQPRQWKDSEVRLMVQVATLSSITIQQVQYQQQLYHRSE

SEQ ID NO: 65 - protein, 197 aa, Unknown
  Unknown Cyanobacteriochrome O6304_4174
REKTISLIIQRMRESLNLETIFQATTNALRQAVDCDRVVIYRFNPDWSGALVSESVSTGWDELLPEQADDPILTQATTDAPGCVLTQMNSSNFLIKDTYLQEQQGGIYRKKQTFCSVSDIYQAGFSSCYLEFLEQLSARAYVIAPIFSGNTLWGLLGVYQNSGPRDWKEAQVKIVTQIGNQLGVAVQQAELFAQTQK

SEQ ID NO: 66 - protein, 197 aa, Unknown
  Unknown Cyanobacteriochrome O6304_4065g2
REKAVASAVQRIRQSLDLGTIFAATTEELQGCLTCDIVAIYRFNPDYSGEFVSEAITSQRVSVLQRNRQSLHLSAQPASGENCQMQALINSCLEWQDTHLIQTKGDAFNRDENCLVVEDIYQRNFSPCYLARLEEFQVRAYIIVPIFCGSKLWGLLAAYESDRPRQWQPSDHKIVIQIGNQLGVALQQAELLEQTQK

SEQ ID NO: 67 - protein, 195 aa, Unknown
  Unknown Cyanobacteriochrome O6304_1286
REKALAAAIQTIRRSLDLDTIFAATTQELRTCIDCDRVAIYQFDMNYQGQFVAESINGDWISFLQQSNNPPQLLSNSESEPYCRIEAMLKSPNLWPDSYLMENPAILQDPDKMCLVVEDVNLANFSSCYQDRLEQFGIRAYVIVSIFCGSKIWGLLAAYQHSGPRKWKASDRNIVMQIGTQLGVALQQAELLEQK

SEQ ID NO: 68 - protein, 196 aa, Unknown
  Unknown Cyanobacteriochrome AFZ15460g3
LIKATERERAAAKVIDRLRQSLDIDTIFKTTTRDVRELLKVDRVGVYRFNDDWSGEFVLESVGAEWTPLLQKSANLEGLQANQSNCHAVQNLGGKRRLADSYLQETEGGRFREKGTFAVEDIYKAGFSPCYMEVLEESQVKAYAIVPVYQGEKLWGLLGAYQNSGTRKWEEAEVALLAQIGDQLGVALQQAEYLQQ

SEQ ID NO: 69 - protein, 514 aa, Unknown
  Unknown Cyanobacteriochrome NpF0020
MEMNLQFPGINLISLKEAPIHISSQIQPHGVLLVLEEPELKILQVSTNTLKVFGIAPENMLHKKLEDLLDPFQIERIKTGLSGENLDFINPTKVWVRKKGDEYVVFDAIFHRNIEGFLILELEPAITQENIPFLSFYHLARASINQLEKTANLRDFCQVIVQEVRKITEFDRVMLYKFDDDGHGSVIAEEKLESLEPYLGLHYPESDIPKPARKLFAANSIRIIPDAYSQPVKLFPVNNPISDRPINLTNSILRSAASCHTEYLHNMGVGASLTISLIKDQKLWGLIACHHQSPKYVSYELRKACEFLGRVIFAEISAREETEDYYHRINLTHLQSILIEYMSQEENFIDGLVKNPQHLLDLASAQGAAVCFAGNCTVVGETPREEDLNFLVQWLKNNVEEEVFYTDSLPQIYPDAESFKNVASGLLAIPIAKRTYVLWFRPEVIQTVNWGGDPNKAFEVSQSEGNVRLCPRKSFELWKETVRLTSLPWKDVEVKAALELRKAIVNIVLRQADE

SEQ ID NO: 70 - protein, 514 aa, Unknown
  Unknown Cyanobacteriochrome NpF1183
MGSQPENTTTQATALTNHDRKPIHIPGSIQPHGILLALSTQLEIVQVSNNTQVYLCKAPEDLLGRPLSYLLEPQPVEIVKQCLVKKVGSANAFKVLINTLYGEIYFDAIAHRTEEAVILELEPTDSEFEVSFLNFHSFASEAIAKMQRTSNLGEFLHLVAQEVQKIISFDRVMVYQFDESEAGSVVAEVKREDLSPYLGLHYPATDIPAQARELYTRCFLRFLPDLTAEPVKLVPTENPTTHQHLDLSYCLLRSFDWCCAEYHQNMGVKALLVISLIQEQKLWGLISCHHQTPKYISYEVRKMCEFLGQIVSSELAHKISSSEWDYKVKLKSLQSEFLESISQADNFIDALIKPEIRLLDFVSASGAAVCLDNEINLVGTTPNIDEVRALIEWADTQVTDNLFSTDSLPKLYPEALIFKDTASGLLLLRISKVRRYYILWFRPEVIQTVHWAGNPQESIKAEGDGSYTLSPRKSFEQWQETVRLTSLPWKGCELESAIALSNAIVGIVLSKADE

SEQ ID NO: 71 - protein, 440 aa, Unknown
  Unknown Cyanobacteriochrome NpR1759
MGLVTNSGFRNLKDQFLPERKEALLARMANRIRQSLNLQEILDATVVELRAFLQTDRTKVYRFDRDGHGHVVAEASAPNRLPSLLGLHYPADDIPPQARALFVKARTRSIVNVSEQRIILNSLPTPTTTAIGDLTVEEVQEQPLEDILSRPVDPCHVDYLTQMGVQSSLVVPIIYQQELWGLLISHHAEPREITSEDLLVVQLLADQVSLAITQASLLSQVQEQQQRDAIVNQIATMLHAPLTPEEILHNVLEQAVKASGSSGGRLYLTSPDEALPAHLYTYGNQPAPSEQEQPHLLENHPLWQEVRKIPAPIGTTLTQKDKPALRVVINLQQEPRLHQLIESFQTTPIRGLIVQPLYYSKEFLGYLTFFRDQISTKNLWPGYEEADERQQRPRQSMAQWQALKEDKAHPWSIEEMELIQSLNIHLSLAVLQNRLYHRER

SEQ ID NO: 72 - protein, 412 aa, Unknown
  Unknown Cyanobacteriochrome NpR5313g1
MGTSTDKPNGLQQSLEQESLLHRMIKQIRRSLDLQEILTTTVTEVRSFLRADRVKVYRFDTSGSGEVIAESIHNERLPSLLGLRFPVHDIPEAAREMFLLAGQRSIVDVANHKIGLSPLQSTETGKHLQTNIYYRKVDPCHIQYLKAMGVQSSLVVPILDSDPQQSAKPKLWGLLVSHQSKPRKILKREIKVLQQVADQVAIAIAQSNLLTAALTQQKQEATINRVTTLLHKLPTIQLQGAIEEVITAFSGVGGRLYIEQSRELYTWGDQPTLPYELDSSIIEQHPTWQNWMTECQPGNIWATSDLYKEPHLRVLALAFRSTQIRGLMVIPLHYREQFIGVLSIFRAEFETEILWAGRCEQNRRQLLPQLSFEIWREQKKGLAPEWKPEDMTLAQALYDHFSMAIQQQQIYK

SEQ ID NO: 73 - protein, 503 aa, Unknown
  Unknown Cyanobacteriochrome NpAF142g1
FLTNQVSQTQEILLHRIASRIRQSLELQEILSATVAEVRSFLGTDRIKIYQFQADGHGLVIAESIQEDRLPPLLGLNFPADDIPPYARELFVRARQRCIVDLTTQEIGISPLDCPETGKPLEQQDIRYRPVDPCHLEYLTAMGVKSSVVVPIVLKNQETGKDSSPNVMESSQLWGLLVSHHSQARVVTQQELLLIQSVVDQVAIAISQSILLTQVREQARQEAIINKVTEQLHSTPVAQLQTALEETVAAFNGSGGRLYLLPDDEQTAKLYTFGLQPNQLDIGQEQTPSPLTQELSLVDRAWVSPMERNNEVLLLANLREGSDVLRKGRPIEEHRLWQKYLFASICPPENLENPSHKSWSVNWMRAIYALTPPVNELSYDSNLWAIADLYKEPLLRSVAPCFQTTQVRGLLIVPLQHGSTIVGCLTIFRDEVDIETIWAGCVDTDSRQLMPRQSFAAWRELRTGQAQQWSESEIKLAQALGERFATAVKQHRLYEQVQALNAN

SEQ ID NO: 74 - protein, 514 aa, Unknown
  Unknown Cyanobacteriochrome Cph1
MATTVQLSDQSLRQLETLAIHTAHLIQPHGLVVVLQEPDLTISQISANCTGILGRSPEDLLGRTLGEVFDSFQIDPIQSRLTAGQISSLNPSKLWARVMGDDFVIFDGVFHRNSDGLLVCELEPAYTSDNLPFLGFYHMANAALNRLRQQANLRDFYDVIVEEVRRMTGFDRVMLYRFDENNHGDVIAEDKRDDMEPYLGLHYPESDIPQPARRLFIHNPIRVIPDVYGVAVPLTPAVNPSTNRAVDLTESILRSAYHCHLTYLKNMGVGASLTISLIKDGHLWGLIACHHQTPKVIPFELRKACEFFGRVVFSNISAQEDTETFDYRVQLAEHEAVLLDKMTTAADFVEGLTNHPDRLLGLTGSQGAAICFGEKLILVGETPDEKAVQYLLQWLENREVQDVFFTSSLSQIYPDAVNFKSVASGLLAIPIARHNFLLWFRPEVLQTVNWGGDPNHAYEATQEDGKIELHPRQSFDLWKEIVRLQSLPWQSVEIQSALALKKAIVNLILRQAEE

SEQ ID NO: 75 - protein, 1276 aa, Unknown
  Unknown Cyanobacteriochrome Cph2
MNPNRSLEDFLRNVINKFHRALTLRETLQVIVEEARIFLGVDRVKIYKFASDGSGEVLAEAVNRAALPSLLGLHFPVEDIPPQAREELGNQRKMIAVDVAHRRKKSHELSGRISPTEHSNGHYTTVDSCHIQYLLAMGVLSSLTVPVMQDQQLWGIMAVHHSKPRRFTEQEWETMALLSKEVSLAITQSQLSRQVHQQQVQEALVQRLETTVAQYGDRPETWQYALETVGQAVEADGAVLYIAPDLTGSVAQHYQWNLRFDWGNWLETSLWQELMRGQPSAAMEPMAAVQSTWEKPRPFTSVAPLPPTNCVPHGYTLGELEQRSDWIAPPESLSAENFQSFLIVPLAADQQWVGSLILLRKEKSLVKHWAGKRGIDRRNILPRLSFEAWEETQKLVPTWNRSERKLAQVASTQLYMAITQQFVTRLITQQTAYDPLTQLPNWIIFNRQLTLALLDALYEGKMVGVLVIAMDRFKRINESFGHKTGDGLLQEVADRLNQKLSPLAAYSPLLSRWHGDGFTILLTQISDNQEMIPLCERLLSTFQEPFFLQGQPIYLTASMGISTAPYDGETAESLLKFAEIALTRAKCQGKNTYQFYRPQDSAPMLDRLTLESDLRQALTNQEFVLYFQPQVALDTGKLLGVEALVRWQHPRLGQVAPDVFIPLAEELGLINHLGQWVLETACATHQHFFRETGRRLRMAVNISARQFQDEKWLNSVLECLKRTGMPPEDLELEITESLMMEDIKGTVVLLHRLREEGVQVAIDDFGTGYSSLSILKQLPIHRLKIDKSFVNDLLNEGADTAIIQYVIDLANGLNLETVAEGIESEAQLQRLQKMGCHLGQGYFLTRPLPAEAMMTYLYYPQILDFGPTPPLPKVALPETETEAGQGNVGDRPLPNSLNRENPWTEKLHDYVLLKERLQQRNVKEKLVLKIANKIRASLNINDILYSTVTEVRQFLNTDRVVLFKFNSQWSGQVVTESHNDFCRSIINDEIDDPCFKGHYLRLYREGRVRAVSDIEKADLADCHKELLRHYQVKANLVVPVVFNENLWGLLIAHECKTPRYWQEEDLQLLMELATQVAIAIHQGELYEQLETANIRLQQISSLDALTQVGNRYLFDSTLEREWQRLQRIREPLALLLCDVDFFKGFNDNYGHPAGDRCLKKIADAMAKVAKRPTDLVARYGGEEFAIILSETSLEGAINVTEALQVEVANLAIPHTVSGTGHVTLSIGIAVYTPERHINPNALVKAADLALYEAKAKGRNQWLAYEGSQLPHVDGEV

SEQ ID NO: 76 - protein, 542 aa, Unknown
  Unknown Cyanobacteriochrome CparGPS1
MEWGDLERCEGARIQSWGCVLVVRPSDQFIVWCSERAFEWLGFELDSLSLEPNGIDIALIVPEARTVLRNDDFNSWRAVAFTTTKGKQFHCSIHTTEEYYLLEFEEYCAESLAKMKDSINEASERILCIDNEEELLTQMIKETSELCSFGDRVLIYHFLPATGGAHGRVMAENLRCGKEAGWQTFKGLNFPSSDVPFSSRKLFQLTRHRYLAAVSGPQIPLVKIAPGVVSPPDLSRSFLRGYTNCCQQYYVNMGVKAILVYAIELPGQGLWGFLSFHSTTENYVPIVKRNALLPLVRTFAGTLKSIKARKRMADESRAKELQLQIFGRLRGQTNFLMGMLDGQPSLKDALPCGSCVVFHDDEVLCGTGPLPPVDFILRTMKMIRSSTNPIESTWMTGSLDPGRLWLTDKLSDLIPEARLISGISAGALLVQVSWRMQVYVMWIRPAVEREVSWAGAPPGSAYSIDKEGDLNPRASFQKWRELVMDRCEPWDESCLNAAQAVGNTMAEALRINKMISSSTKRLNMLAEQMHQIISNVPVAVFR

SEQ ID NO: 77 - protein, 548 aa, Unknown
  Unknown Cyanobacteriochrome GwitGPS1
MEVDEHTVDTDESHTTPLEFANCIQPHGILFAVRRKDPNLTILQVSKNVWELIGVKVEDVLGAPFLNLVKPKYRDALFSVLNGSDDVSMSGQRPPFPLRMISNNSKWSCIFYPTDSGIVVELEPVSHASDDPTQLSFKSSAAGLMKVNAAIERMKSATDVHQLFEISAKELRTMLGFDRVLLYCLSASGHGRVVAESISDQVKDRVTLYGAQFPARDIPQNAHCMFTTKMQRLIVDIDQDPSPLVPERNPDTHRILDLSRSVLRGTMSCHIRYVRNMLVKANFVNGIPKGDGKLWGLLIAHHFSAKFVPYESRASAAQLILALSVLIDNKGEEEYRHFEKQATAVQIQMLKLAPKEPAEDIMARMTPLLRDVMKCDGLAFVTPQFSVNVGNCPPKASMFAIAEYLTANDEAISGLWETNCTDNIPTLKGLDLAGCCGILALTISSINKEFLLWFRGAVKHHVRWSTVSTNEANPLAHIGNREAFVVWRQAVTNCSESWAIADLAVARTLRLAISDVRTTNEMRLKARMLEEMNRELANSNRQISRVAT

SEQ ID NO: 78 - protein, 524 aa, Unknown
  Unknown Cyanobacteriochrome EsPHL1
MEMSHSCSGDPERLAQIGHIQGCGYLFAVQETTTGYPSGLRIVTVSQNIMEAPWVQASSESDILGKDLGDMLREECVQTVRALVTRYTQASQPRNDRISPKVNRTTADIYSCPAAAHAGLVPGEEDDPTKAVFTFTLTGSNPGVYLVEVERKHGPHSARAQHTPGLLQMTDLMGCIPVGSNSLVSTAALCDALAENMPAYERVVVYRFAPDGHGEVIHECIKPGCEVESSFLGCHFPASDIPPIARKLLMLKGVSFIADTSGQRVAIRSIPERSSTPLDLSRSALRAPSPCHEQFLRNMGVRASMAVAITVGGELWGNINFHAYTRAVHPSCEERILAETTVTLTGALIMHYERQEIATTALALSRMLGRLKDYPRVNDFLSSEHQALRDILEVDTIVLYEHSRSATVYGKKDISLTLSECIELLNGDGPGETLCFRSLEAKGVAFFSVNTFLIVFLRENIADQIKWAGNPEAIMHDGEVVRPRASFQQFMGTPAVPFKLWSPATTDLLNMVRRGFSSKIYADA

SEQ ID NO: 79 - protein, 649 aa, Unknown
  Unknown Cyanobacteriochrome DtenPHY1
MAAKISRSTPAINGNLVAQKQSLRDAVLEEEWREGARAAGGNEGAMAKGYDYAGRIRELRAAEERAREGGDQAVATHSQIEKYTASIFRPGGVQPHGCCVVFDPEMNRIVALSRNAPEKLGVSIAPDSMLGRSPSELFGQRTLGILAHAAKSDAVAMDLLCPFPVQSKDERDSGMQASLHSTDEGLVMDIEPVDSGVQGLMACHRLIIASAERLRMQSTPSELFQTMAEEVHALTKYNRVMIYKLHPDAHGEVIAESVDAGVSSFLGLHFPGTDIPQVNRTLFECLPKPRLLVDACAPSSEMVQEEHLKQGVLLGASQLRGTVGCHAEYLKNMKVTGTLVTPVTVVDQEASTQRRLWGLIVCHHEQERFVGYEYRAALSLLIQIFTVRLEKLLQVEAELNKQSVSSAQNKLMDILLSDPSLRAHELLFGPTAVPELLAIVPGDALCIVSPNTGRVATGGEYPPSDDLALRLAQWLWSWYKSKRASREKDAMRVFSIPSLAAEGFPEAKDVAETVAGVLVVVLQLQSGSTTPLATTSDRSEDSSSVLIVWTRREFKEIITWAGGTDKTNYVQHSAGGSLHPRGSFGIITEEYALTAHPWLKQELDTVQQLGILLQETTESNEASSLRMDILSSLNEQRFKGMQEVTRLAA

SEQ ID NO: 80 - protein, 571 aa, Unknown
  Unknown Cyanobacteriochrome NpyrPHY1
MEDGATRYGLCDDDAKDVELSKLYEQQATIALASSKAKLDEEIRDFEAYGRLINRPGLIQAHGCTILLQKKTLKILASSMNSLDHLGVFLDADSLQDIQVCMQAADPTLTNPRHVKTVSGKTLHAILHHVKDGTATGVTACFRMIHEAMERIGVIKDLSTLYDGIVKEIQNLSGYDRVMLYQFHEDNHGEVVAECRKDVNIPSYLNLHFPATDIPQVSRRLFMTMKQRMVGDIKASQVGFVQAPELAEENLLLSQSQLRGVTGCHAQYLENIGVRATLVNAVVVGPTHSSAASAFGTKDNSRKLWGLIVCHNLHDKRIVEFHVRSALLFLVDVFALQLQRLMDIMETAQNELMWSAQNELYNNVVTTDDFNIGAVADNPECLMKFVGASGAAVLSGGSLHLIGRTPRAVQLNELADWIRAQPSYDDQRVVCTRSLVADGAPGAASYVQDAAGLLAVGVPQKATSSVPDSMIIWFRSEVVESVTWAGTNEQHVPKRESDYLQPRESFKAYLKTMANTSARWEPRILVSVRSFQEVLSLTQTCDNKMKTLAHMTSRVSVQRSITVSQLTAVSR

SEQ ID NO: 81 - protein, 776 aa, Unknown
  Unknown Cyanobacteriochrome PcolPHY1
MERAGACRARAELAPPAAGEGRAPGLYGARRGAHGRFVFCGRRGLIAVFPADGADAAERARLVEKVTERIYRPGVVQGHGAVLILERAHALAFGAHTLAAAPKGGKGQSGGRAGPTARPPAPRVIAASETCAPMLGVGASELLGAPASRALTSRSLRKICAAAASGAPEARTPIAIAPREADARRRSKEHGGALAAAVHVTEEGVVIDLESTHTSPLAPLPGCEANADGSHTGAAVGATFGGCGSDSDSDSDSSADDVRDLPPGVSQCHRAAAGAAMRLRALRGEATLCEALACEVREISGFDRCMVYRLHADMHGEVCAEARRAESVPSFLHLHWPATDLPQVNRRLLTKHPAPRLLADVNDTPSPVLQCAEGDLASAFGCRQALLAGSQIRGTIGCHKEYLNNMGVRGTLVVPLIIAAAAADSADDAHEDIGGSSSAGGGKPALRMWGMVVCHNISPRFVPLHTRVCLSMIAQIFTQRLEHELEIARLSTADRNARANSRLVELIIADNPRQKATEVLSGSLHTLREAVPFDADGVFVLDSSCTVIACEGAAESGRIEAARVARWIKSSGKLHEDAQHPGVFVCDSLEVAGYAALAAAQAVAGGIAGVVAIELPAGVTGTDGHNGASALEEGACSDPEIAESGGSAVGSPKGRRAIGGMIIWTRAELLQDIKWAGARGDSRDVVVIDHDDGAGDDDVKGAPRLGPRSSFAVVLEQLRGTCELWPRIAARGAGALVAVLRDAVEAATGSLLRAGIASVLNRSRLSDMGELEKVAA

SEQ ID NO: 82 - protein, 680 aa, Unknown
  Unknown Cyanobacteriochrome TastPHY1
MGDAAGRARVRAQDRQFLADVELEARFALTDGGCPASAQQPAASAGFNYQDSVEVTKAANSSAARGIPVSTDQLHAYDRRTLRPGQVQSFGALLLVGHGESQGKCAFPRGLVLAASANSKEVLGVEASQLLDTFLFNPEVSPFDAECLGALDELLKSRDLTAQAPLLATLRLVGGSGAQRQAFLIVHSTEEGYCIDVEPLNEDVSRLLRGSILTHSLAKKGVERLQEMSSCSVEEQCQALCEEARALTGYDRIMVYKFHPDCHGEVVAESVSEHVKEPMLGLHFPATDIPQANRGIFMAMRSRMIADTSASNVKILQSPRLLDNIILAKSQLRSVSGCHATYLQNMGSTATLTLSIVTAPTADGFQLKPNYFKMGLPDLSGAKENEGRPPPRSPLWGLVCCHHSQGPYRVNYDHRSAAEFLVKVFSLQLGRRLDALEAAAQDRVNIAQQSICKVLKSIEESPHHLEGRTEALADGLMRGHSGKVMMQVAEATGAAMLVGGKWRTTGLCPQPDQLDTLAAWLCSGQEQAPPRLPHGGRWGTISLHKEGFPNAKAIRGCAAGLLALDLSRSAASSTGTQCLAIWFRGELMKEQNWAGNKNDPQGRAMGHEMTPRSSFIAMSEILDLECRQWADCEVDGTHTIQLLLQDSIRFAEEGMTTGRILIAINQERLRNMDELTKVAS